(19) United States
(12) Patent Application Publication
(10) Pub. No.: US 2023/0151409 A1
(43) Pub. Date: May 18, 2023

(54) METHODS AND COMPOSITIONS FOR NONINVASIVE PRENATAL DIAGNOSIS THROUGH TARGETED COVALENT LABELING OF GENOMIC SITES

(71) Applicant: Vilnius University, Vilnius (LT)

(72) Inventors: Edita Kriukiene, Vilnius (LT); Juozas Gordevicius, Vilnius (LT); Milda Narmonte, Vilnius (LT); Povilas Gibas, Vilnius (LT)

(21) Appl. No.: 17/916,056

(22) PCT Filed: Mar. 30, 2020
(86) PCT No.: PCT/IB2020/053011

§ 371 Date: Sep. 30, 2022

(51) Int. Cl. C12Q 1/6827; C12Q 1/6853; C12Q 1/686

(52) U.S. Cl. CPC C12Q 1/6827; C12Q 1/6853; C12Q 1/686; C12Q 2600/154; C12Q 2600/118

(57) ABSTRACT

This invention relates to a method that covalently modifies unmodified and hydroxymethylated genomic sites in fetal specific genetic material present in maternal blood DNA samples and produce the adjacent genomic regions for detecting fetal aneuploidies and fetal gender using quantitative real time PCR or sequencing. A large panel of differently labeled sites and regions between maternal and fetal genetic material has been identified and they validity for diagnostic purposes of fetal trisomy of chromosome 21 has been demonstrated.

### TECHNICAL FIELD

This invention relates to the field of genetic testing for pregnant females in order to diagnose chromosomal aneuploidy and fetal gender from maternal peripheral blood samples.

### BACKGROUND ART

Fetal chromosomal aneuploidy results from the presence of abnormal dose(s) of a chromosome or chromosomal region. The Down syndrome or Trisomy 21 (T21) is the most common incurable chromosomal aneuploidy in live born infants, which is typically associated with physical and mental disability (Parker et al. 2010). The overall incidence of T21 is approximately 1 in 700 births in the general obstetrical population, but this risk increases to 1 in 35 term births for women 45 years of age. An invasive diagnostic procedure is currently the only way to confirm the diagnosis of T21, commonly by a fetal cytogenetic analysis (such as karyotyping), which requires fetal genetic material to be invasively obtained by amniocentesis, chorionic villus sampling or cordocentesis. Due to the current risk of prenatal testing it is currently offered only for women in the high-risk group. Although the safety of invasive procedures has improved since their introduction, a well-recognized risk of fetal loss (0.5 to 1% for chorionic villus sampling and amniocentesis) and follow-up infections still remain (Akolekar et al. 2015). Hence, non-invasive and highly confident prenatal screening tests to reduce the number of invasive diagnostic procedures are still required.

Since the discovery of fetal genomic material in the form of circulating cell-free fetal DNA (cffDNA) in the blood plasma of pregnant women (Lo, et al., 1997) many attempts have been made aiming at using cffDNA for non-invasive risk-free prenatal testing (NIPT). Early applications of NIPT included the determination of Rhesus D blood-group status and fetal sex as well as the diagnosis of autosomal dominant disorders of paternal inheritance by quantitative real time PCR (qPCR) (Lo et al., 1998; Daniels et al, 2006). However, the application of cffDNA to the prenatal detection of fetal chromosomal aneuploidies has represented a considerable challenge. First of all, the cffDNA represents only a subfraction of 6-10% of the total cfDNA (cell-free DNA) of maternal origin in first and second trimester pregnancies and rises up to 10-20% in third trimester pregnancies (Lun et al., 2008; Lo et al., 2010), and this can often interfere with the analysis of fetal nucleic acids. One way to deal with the low abundance of the fetal DNA was the evaluation of the dosage of chromosome 21 calculating the ratios of polymorphic alleles in the placenta-derived DNA/RNA molecules (Lo, and Chiu, 2007). However, this method can only be applied to fetuses that are heterozygous for the targeted polymorphisms.

A study of Zimmermann et al (2002) was able to distinguish between trisomic 21 and euploid fetuses using qPCR based on the 1.5-fold increase in chromosome 21 dosage in the trisomic cases. Since a 2-fold difference in DNA template concentration constitutes a difference of only one threshold cycle (C_{T}), the discrimination of a 1.5-fold difference is at the limit of conventional qPCR.

With the development of massive parallel sequencing (MPS) the detection of fetal aneuploidy is carried out through counting cfDNA molecules and measuring the over- or underrepresentation of any chromosome in maternal plasma. As previous reports have indicated that fetal cffDNA is shorter than its maternal counterpart (Chan et al, 2004; Li et al, 2004; Fan et al, 2010), MPS has been combined with size fractionation prior to sequencing or in silico of plasma DNA fragments to enrich for fetal DNA. However, even though MPS has been widely used in commercial prenatal testing, such an approach which requires deep coverage or paired-end sequencing, increases the cost of service.

An alternative approach to improve the sensitivity and cost-effectiveness of NIPT is preferential targeting of fetal DNA sequences by utilizing epigenetic differences between maternal blood DNA and cffDNA.

Bisulfite conversion that enables analysis of the methylation status of each CG site, followed by either methylation-specific PCR or sequencing has been applied to detect methylation differences between maternal and fetal DNA (Chim, et al. 2005; Chiu, et al. 2007; Chim, et al. 2008; Lun et al, 2013; Jensen et al, 2015). However, although providing high resolution, bisulfite treatment reinforces the degradation of low amounts of fetal DNA, complicating fetal specific methylome analysis. Furthermore, screening genomes for diagnostic of DMRs by whole-genome bisulfite-sequencing is technologically demanding and extremely expensive leading to an unnecessary increase in cost of NIPT.

The application of methylation sensitive restriction digestion involves the use of methylation-sensitive restriction enzymes to remove hypomethylated maternal DNA thus allowing direct polymerase chain reaction (PCR) analysis of cffDNA (Old, et al. 2007; Tong et al, 2010). However, methylation sensitive restriction digestion is inherently limited by the sequence-specificity of available enzymes what restricts the number of DMR regions suitable for testing.

The methylcytosine-immunoprecipitation based approach (MeDIP) was used in combination with oligonucleotide array analysis, sequencing and MeDIP-qPCR for the quantification of selected hypermethylated fetal DMRs on chromosome 21 (Papageorgiou et al., 2009, Tsaliki et al, 2012, Keravnou et al, 2016). However, MeDIP enrichment is biased to highly methylated sequences (Weber et al. 2005) and thus, the potential diagnostic informativeness of the less CG dense or less methylated sequences might be lost. Therefore, further developments and advances are necessary for the identification and detection of highly specific and stable fetal-specific markers.

Placental DNA was reported to be generally hypomethylated as compared to maternal blood DNA. Examination of the differential methylation between placenta and maternal blood uncovered large contiguous genomic regions with significant placental hypomethylation relative to non-pregnant female cfDNA (Jensen et al, 2015). Moreover, these regions are of low CpG and gene density and thus could be poorly covered by affinity enrichment methods, such as MeDIP. Since unmodified CG fraction represents smaller portion of the human genome (20-30% of CGs are unmethylated), its targeted analysis is more relevant for cost-effective and sensitive detection of fetal specific DNA fragments in maternal circulation.

In recent years, we and others have been adapted covalent derivatization for epigenome-wide studies of various cytosine modifications (Song et al. 2011; Kriukienė et al. 2013; Staševskij et al. 2017; Gibas et al, 2020, accepted). Generally, robust and highly specific enrichment of a covalently modified minor fraction of cytosines in the fetal cffDNA, for example of unmodified CGs or hydroxymethylated cytosines, could potentially help achieve superior sensitivity and specificity in prenatal diagnostics. More importantly, a method for highly specific targeted analysis of a particular fraction of fetal regions combined with lower cost next generation sequencing devices or real time quantitative PCR (qPCR) can significantly alter the cost and turnaround time of NIPT, increasing the availability of NIPT screening for all pregnancies without the restriction to a high risk group.

### SUMMARY OF INVENTION

In the first aspect, the present invention provides a new method for noninvasive prenatal diagnosis based on analysis of unmodified CG sites (uCG) or hydroxymethylated CGs (hmCGs) in nucleic acid molecules extracted from a biological sample obtained from a pregnant female typically during the first trimester of gestational age through use covalent modification of uCGs or hmCs and subsequent estimation of the labeled fraction of CG sites, enabling genome-wide identification of the fetal-specific regions.

According to one exemplary embodiment, a biological sample received from a pregnant female is analyzed to perform a prenatal diagnosis of a fetal chromosomal aneuploidy, such as trisomy T21, and fetal gender.

A maternal biological sample includes nucleic acid molecules found in various maternal body fluids, such as peripheral blood or a fractionated portion of peripheral blood, urine, plasma, serum, and other suitable biological samples. In a preferred embodiment, the maternal biological sample is a fractionated portion of maternal peripheral blood.

A large number of differentially labeled regions (DLRs) on chromosome 21, 13 and 18 which are differentially modified between non-pregnant female peripheral blood DNA sample and DNA of placental origin (chorionic villi (CV) of the fetal part of placenta which are enriched in fetal trophoblasts) or between non-pregnant female peripheral blood DNA sample and peripheral blood DNA sample of pregnant women have been identified using covalent chemical modification of the cytosine base of naturally unmodified CG sites or hydroxymethylated CG sites in maternal nucleic acid molecules. Subsequent PCR amplification with or without enrichment of the labeled fraction of CG sites coupled with sequence determination of the labeled and amplified nucleic acid molecules enabled genome-wide identification of the fetal-specific labeled regions. As used herein, the term DLR refers to a “differently labeled genomic region” that is more or less intensively labeled through enzymatic transfer of a reactive group onto the cytosine base in the nucleic acid molecule. For the purposes of the invention, the preferred DLRs (selected u-DLRs; see Table 4) are those that are hypomethylated and thus, more intensively labeled, in fetal DNA and hypermethylated in maternal DNA. In another aspect, the preferred DLRs (selected hm-DLRs; see Table 5) are those that are hyper-hydroxymethylated and thus, more intensively labeled, in fetal DNA and hypo-hydroxymethylated in maternal DNA.

In one embodiment, a DLR can be confined to a single cytosine or a dinucleotide, preferentially a CG dinucleotide (CG-DLRs).

Representative examples of a subset of these u-DLRs, hm-DLRs and CG-DLRs have been used to accurately predict trisomy 21, in a method based on analysis of fetal-specific hypomethylated or hydroxymethylated DNA in a sample of maternal blood, typically during the first trimester of gestational age. Thus, the effectiveness of the disclosed DLRs and methodologies for diagnosing fetal aneuploidies have been demonstrated.

In addition, representative examples of a subset of these u-DLRs and hm-DLRs have been used to accurately predict fetal gender from X and Y chromosomes, in a method based on analysis of fetal-specific hypomethylated DNA in a sample of maternal blood, typically during the first trimester of gestational age. Thus, the effectiveness of the disclosed DLRs and methodologies for diagnosing fetal gender have been demonstrated.

Accordingly, the invention pertains to a method for prenatal diagnosis of a trisomy 21, and fetal gender using a sample of maternal blood, the method comprising:

(a) enzymatic labeling of uCG and hmC sites of nucleic acid molecules in a sample of maternal blood with a first reactive group, preferably an azide group;
(b) chemically tethering of an oligodeoxyribonucleotide (ODN) having the second reactive group, preferably an alkyne group, to the first group in a template nucleic acid;
(c) producing nucleic acid molecules from a template nucleic acid sequence using a nucleic acid polymerase which contacts a template nucleic acid sequence at or around the site of the labeled uCG/hmC and starts polymerization from the 3′-end of a primer non-covalently attached to the ODN;
(d) determining the presence or availability of the CG target sites and hence the level of the unmodified or hydroxymethylated template genomic nucleic acid molecules across the regions of chromosomal DNA shown in Tables 4 or 5, or 6;
(e) comparing the acquired value of the regions of step (d) to a standard reference value for the combination of at least one region from the list shown in Tables 4-6, wherein the standard reference value is (i) a value for a DNA sample from a woman bearing a fetus without trisomy 21; or (ii) a value for a DNA sample from a woman bearing a fetus with trisomy 21.
(f) diagnosing a trisomy based on said comparison, wherein trisomy 21 is diagnosed if the acquired value of the regions of step (d) is (i) higher than the standard reference value from a woman bearing a fetus without trisomy 21; or (ii) lower than the standard reference value from a woman bearing a fetus without trisomy 21; or (iii) comparable to the standard reference value from a woman bearing a fetus with trisomy 21.
(g) detecting fetal gender based on said comparison wherein female gender of a fetus is detected if the acquired value of the regions of step (d) is comparable to the standard reference value from a woman bearing a female fetus, and male gender of a fetus is detected if the acquired value of the regions of step (d) is comparable to the standard reference value from a woman bearing a male fetus.

### BRIEF DESCRIPTION OF DRAWINGS

FIG. 1 is a diagram of the methodology for identification of Differentially Labeled Regions (DLRs) across chromosome 21 (or chromosomes 13 and 18) comparing the two tissue pairs: chorionic villi tissue DNA of the 1st trimester fetuses and fractionated peripheral blood DNA samples of non-pregnant controls and fractionated peripheral blood DNA samples of non-pregnant female and pregnant female carrying a healthy fetus from the 1st trimester pregnancies. Further strategy for area under curve (AUC) determination for diagnosing T21-affected fetuses is also shown.

FIG. 2 shows the difference in (a) uCG and (b) hmCG signal for the exemplary DLRs (tissue-specific u-DLR chr21:33840400-33840500; pregnancy-specific u-DLR chr21:33591700-33591800; tissue-specific hm-DLR chr21:35203200-35203300; pregnancy-specific hm-DLR chr21:43790900-43791000, selected from Tables 4 or 5) identified in chromosome 21 between chorionic villi tissue DNA of the 1st trimester fetuses and fractionated peripheral blood DNA samples of non-pregnant controls; and between fractionated peripheral blood DNA samples of non-pregnant female and pregnant female carrying a healthy fetus from the 1st trimester pregnancies (left panel). For diagnosing purposes of trisomy 21, the signal intensity across the exemplary DLRs is also shown for the samples of pregnant female carrying T21-diagnosed fetuses from the 1st trimester pregnancies (right panel).

FIG. 3 shows the difference in (a) uCG and (b) hmCG signal for the exemplary DLRs (u-DLR chr21:43933400-43933500; hm-DLR chr21:36053400-36053500; selected from the Tables 4 or 5) identified in chromosome 21 between fractionated peripheral blood DNA samples of pregnant female carrying a healthy fetus or a T21 diagnosed fetus from the 1st trimester pregnancies.

FIG. 4 shows the difference in mean signal of labeled individual CG-DLRs, namely, (a) u-CG-DLRs and (b) hm-CG-DLRs (selected from Table 6) in chromosome 21 for detection of fetal T21 aneuploidy.

FIG. 5 shows the difference in mean signal of labeled individual CG-DLRs, namely u-CG-DLRs (selected from Table 6) in chromosome X for fetal gender determination. Samples from pregnant women and fetal CV tissue were labeled either XX or XY according to the gender of a fetus, Female and Male, respectively. Samples from non-pregnant women, NPC, were labeled as None, 00.

FIG. 6 shows the relative quantification of individual or a combination of (a) u-CG-DLRs and (b) hm-CG-DLRs of fetal specific DNA regions located on chromosome 21 using real time quantitative PCR for replicated DNA samples of peripheral blood plasma DNA of women pregnant with healthy or T21-diagnosed fetuses. Y-axis indicates the threshold cycle values (C_{T}) calculated in qPCR for the regions selected from Table 6 whose genome coordinates are shown above the graphs. Notably, numerical values of C_{T} inversely correlate to the abundance of the DLR region, indicating higher abundance of the region in the blood samples of pregnant female carrying a T21-diagnosed fetus.

FIGS. 7a and b show simulation of a PCR-based test for fetal gender determination by measuring DNA methylation differences in (a) chromosome X or (b) chromosome Y, according to the scheme shown in FIG. 8c. DNA of the 1st trimester CV tissue of both genders was mixed with nonpregnant female peripheral blood plasma DNA to the ratio 20/80 or 0/100, respectively, and the difference in the threshold cycle was evaluated by qPCR. ΔC_{T} indicates the difference in the threshold cycle values between the mixtures using the CV samples of both genders (indicated as XX and XY for female and male genders, respectively). FIG. 7c shows relative quantification of fetal specific DNA regions located on chromosome X for fetal gender determination using qPCR for the replicated DNA samples of untreated, i.e. non-preamplified, pregnant female peripheral blood plasma, according to the scheme shown in FIG. 8c.

FIG. 8 is a schematic illustration of the analytical approach for calculation of DLRs using labeling and enrichment of unmodified CG or hydroxymethylated CG sites coupled with analysis by (a) real time quantitative PCR of pre-amplified samples; (b) sequencing of labeled CGs; (c) real time quantitative PCR of non-preamplified DNA samples, of fractionated peripheral blood DNA of pregnant female. ODN—the attached deoxyribonucleotide, A1/A2—the two strands of the ligated to DNA fragments partially complementary adaptors.

FIG. 9 shows the difference in (a) uCG and (b) hmCG signal for the exemplary DLRs (selected from Table 7; the genomic coordinates are shown above the graphs) identified for chromosome 13 and chromosome 18 between CV tissue DNA of the 1st trimester fetuses and fractionated peripheral blood DNA samples of non-pregnant controls; and between fractionated peripheral blood DNA samples of non-pregnant female and pregnant female carrying a healthy fetus from the 1st trimester pregnancies.

FIG. 10 shows the relative quantification of (a) u-CG-DLRs and (b) hm-CG-DLRs of T21 fetal-specific DNA regions located on chromosome 21 using real time quantitative PCR for an independent group of peripheral blood plasma DNA samples of women pregnant with healthy or T21-diagnosed fetuses. Y-axis indicates the threshold cycle values (C_{T}) calculated in qPCR for the regions selected from Table 6.

### DESCRIPTION OF EMBODIMENTS

In the present embodiment, the method comprises the measurement of the presence or availability of the target CG sites in the template nucleic acid molecules by sequencing of the amplified nucleic acid molecules of the biological sample, such that only the sequence of the targeted CGs and hence the unmodified/hydroxymethylated fraction of CGs is determined. In this embodiment, amplification prior to sequencing is performed through the ODN-directed and ligation-mediated PCR using one primer bound complementary to the ODN or a part of it in the absence of complementarity to the genomic template region, and the second primer bound through non-covalent complementary base pairing to oligonucleotide linkers ligated to both ends of the template nucleic acid molecule. In another aspect of this embodiment, amplification prior to sequencing can be performed by targeted PCR amplification utilizing one primer bound complementary to the ODN or a part of it in the presence (5-7 nucleotides complementarity to the genomic template DNA in the proximity of a CG site) or absence of complementarity to the genomic template DNA, and the second primer bound through non-covalent complementary base pairing to the template DNA in the chromosomal regions shown in Tables 4 or 5 or 6 or 7.

In further embodiments, the method comprises the measurement of the presence or availability of the labeled target sites and hence the level of the unmodified or hydroxymethylated template nucleic acid molecules by real time quantitative polymerase chain reaction (qPCR) of the enriched fetal CGs and DNA regions, which have been previously covalently targeted and pre-amplified using attached ODN as described above, utilizing one primer with its 5′ end bound complementary to the chromosomal regions shown in Tables 4-7 in the very close vicinity (its 5′ end binds at or more than 5 nucleotides to a labeled CG site) to the labeled cytosine, and the second primer bound complementary to the template DNA in the selected chromosomal regions shown in Tables 4 or 5 or 6 or 7.

In yet another aspect, the method comprises the measurement of the presence or availability of the labeled target sites and hence the level of the unmodified or hydroxymethylated template nucleic acid molecules in a non-preamplified DNA sample by real time quantitative polymerase chain reaction, utilizing one primer that recognizes and binds to the ODN and 5-7 nucleotides adjacent to the target CG site in a template genomic DNA through non-covalent complementary base pairing, and a second primer binds complementary to the template DNA in the selected chromosomal regions shown in Tables 4 or 5 or 6 or 7.

In the preferred embodiment of the invention, the plurality of differentially labeled regions (DLRs) preferably is chosen from the lists shown in Tables 4-7. In various embodiments, the levels of the plurality of DLRs are determined for at least one DLR, for example chosen from the lists shown in Tables 4-7. Preferably, the levels of the plurality of DLRs in the labeled DNA sample are determined by real time quantitative polymerase chain reaction (qPCR). As used herein, the term “a plurality of DLRs” is intended to mean one or more DLRs (or CG dinucleotides).

In a further aspect, the present invention pertains to a kit, comprising the composition of the invention. In other embodiments, the kit further comprises:

(a) an enzyme capable of covalent derivatization of the cytosine base with an active group, preferentially an azide group;
(b) a compound comprising the active group (an azide group);
(c) an ODN attached to the second reactive group, preferably an alkyne group; and
(d), oligonucleotide primers (e.g., two or more) for assessment of DLR regions through PCR amplification, wherein one primer binds to the ODN or in the close vicinity to the ODN attachment site through non-covalent complementary base pairing and is able to prime a nucleic acid polymerization reaction from the labeled CG and the second primer binds to the genomic regions described in Tables 4-7;
(e) in another embodiment, the kit can further comprise oligonucleotide linkers for ligation and/or oligonucleotide primers for PCR amplification of the nucleic acid molecules to be analyzed by qPCR or sequencing.

### DETAILED DESCRIPTION OF THE INVENTION

The present invention is based, at least in part, on the inventors' identification of a large panel of differentially labeled regions (DLRs) and CGs (CG-DLRs) that exhibit strong labeling in fetal DNA and weak or absence of labeling in maternal DNA. Still further, the invention is based, at least in part, on the inventors' demonstration that hypomethylated/hydroxymethylated fetal DNA can be specifically targeted and enriched through covalent modification of CGs, thereby resulting in a sample enriched for fetal DNA. Still further, the inventors have accurately diagnosed trisomy 21 and fetal gender in a panel of maternal peripheral blood samples using representative examples of the DLRs disclosed herein, thereby demonstrating the effectiveness of the identified DLRs and disclosed methodologies in diagnosing fetal aneuploidy T21 and fetal gender.

Various aspects of this disclosure are described in further detail in the following subsections.

I. A Method for Non-Invasive Detection of Fetal Aneuploidy T21 and Fetal Gender

Accordingly, the invention pertains to a method for prenatal diagnosis of a trisomy 21, and fetal gender using a sample of maternal blood, the method comprising:

(a) enzymatic labeling of uCG or hmC sites of nucleic acid molecules in a sample of maternal blood with a reactive azide group;
(b) chemically tethering of an oligodeoxyribonucleotide (ODN) having an alkyne group to the introduced azide groups in a template nucleic acid;
(c) producing nucleic acid molecules from a template nucleic acid sequence starting at the azide-labeled CG sites through PCR amplification;
(d) determining the labeling intensity level of unmodified or hydroxymethylated template genomic nucleic acid molecules across the regions or CG sites of chromosomal DNA shown in Tables 4 or 5, or 6 using, preferably qPCR, or sequencing of labeled genomic fraction;
(e) comparing the experimentally acquired value of the regions of step (d) to a standard reference value for the combination of at least one region, or at least two regions from the list shown in Tables 4-6, wherein the standard reference value is (i) a value for a DNA sample from a woman bearing a fetus without trisomy 21; or (ii) a value for a DNA sample from a woman bearing a fetus with trisomy 21.
(f) diagnosing a trisomy 21 based on said comparison, wherein trisomy 21 is diagnosed if the experimentally acquired value of the sample is (i) higher than the standard reference value from a woman bearing a fetus without trisomy 21; or (ii) lower than the standard reference value from a woman bearing a fetus without trisomy 21; or (iii) comparable to the standard reference value from a woman bearing a fetus with trisomy 21.

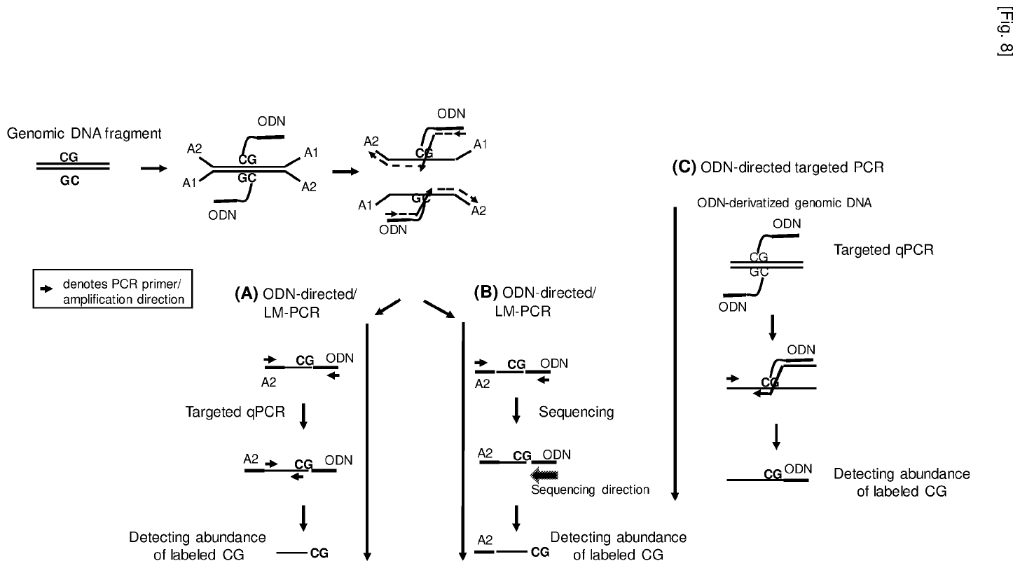

A schematic illustration of the analytical approach for evaluation of labeling intensity in DLRs using labeling and enrichment of unmodified or hydroxymethylated CGs is demonstrated in FIG. 8.

II. Labeling of Unmodified or Hydroxymethylated CG Sites

Methods for the first step of covalent derivatization of genomic DNA sites are known in the art. Covalent labeling of genomic uCG or hmC sites can be performed using an enzyme capable of transfer of a covalent group onto genomic DNA. The enzyme may comprise a methyltransferase or a glucosyltransferase.

An enzyme for covalent labeling of uCG sites is preferably the C5 DNA methyltransferase M.Sssl or a modified variant of it, such as M.Sssl variant Q142A/N370A (Kriukiene et al., 2013; Stasevskij et al, 2017) which is adapted to work with synthetic cofactors, such as Ado-6-azide cofactor (Kriukiene et al., 2013; Masevicius et al., 2016).

An enzyme for covalent labeling of hmC/hmCG sites is preferably the phage T4 beta-glucosyltransferase (BGT) which is adapted to work with synthetic cofactors, such as UDP-6-azidoglucose (Song et al, 2011).

The ODN is preferably from 20 to 90 nucleotides in length, as shown in the exemplary embodiment preferably 39 nt. The ODN contains the reactive group at the second base position from its 5′-end, preferably the alkyne group, which reacts with the azide group which was enzymatically introduced in a template nucleic acid molecule.

It should be noted that DNA after covalent labeling becomes enzymatically and chemically altered but preserves base specificity. As used herein, the term “enzymatically altered” is intended to mean reacting the DNA with an enzymatically transferred chemical group that enables the conversion of respective CG sites into the azide-CG sites, giving discrimination of the labeled sites from template CGs. As used herein, the term “chemically altered” is intended to mean enzymatic transformation of template cytosine into the azide-modified cytosine in CG sites. Thus, in the instant method the fetal specific regions are calculated between more intensively and less intensively labeled CG sites in DNA without the need to directly determine methylation or hydroxymethylation levels of template DNA. Furthermore, in the instant method, the DNA of the maternal blood sample is not subjected to sodium bisulfite conversion or any other similar chemical reactions that alter base specificity, such as sodium bisulfite conversion, nor the maternal blood sample is treated with a methylation-sensitive restriction enzyme(s) or through direct or indirect immunoprecipitation to enrich for a portion of maternal blood sample DNA.

Alternatively, the ODN-derivatized template DNA can be enriched on solid surfaces using an affinity tag that is introduced in the composition of the ODN. A useful affinity tag preferably is but not restricted to the biotin and can be used in the methods of the present invention. In this aspect, the invention includes an additional step of separating maternal nucleic acid sequences on a solid surface, for example on streptavidin/avidin beads, thereby further enriching for nucleic acid molecules containing labeled CG sites. Other approaches known in the art for physical separation of components can be also used. The captured DNA is to be used for further analysis without detachment or can be detached from beads in mild conditions, such as, for example pure water and heating to 95° C. for 5 min.

III. Producing of Template Nucleic Acid Molecules from the Site of Covalent Labeling

In the diagnostic method, a nucleic acid polymerase primes polymerization of the template nucleic acid at or around the site of labeling using the 3′-end of an externally added primer which is non-covalently attached to the ODN. Non-covalent bonding preferably involves base pairing interaction between the ODN and the externally added primer. In the preferred embodiments shown in FIGS. 8a and b, the structure of the ODN permits correct positioning of the externally added primer to the template at the site of the ODN attachment; the primer should be complementary to the sequence of the ODN while should not make any complimentary base pairing with the template nucleic acid at its 3′-end. In yet another aspect, shown in FIG. 8c the primer at its 5′-end should be complementary to the sequence of the ODN while its 3′-end should make complementary base pairing with preferably at least 5 nucleotides and not more than 7 nucleotides of the template nucleic acid that are adjacent to the site of the attached ODN.

In the diagnostic method, typically after tagging of CGs in the maternal blood sample with the ODN, the tagged CGs and adjacent template nucleic acid are pre-amplified starting from the site of the attachment of the ODN. As used herein, the term “pre-amplified” is intended to mean that additional copies of the DNA are made to thereby increase the number of copies of the DNA, which is typically accomplished using the polymerase chain reaction (PCR).

In the preferred embodiment, the experimentally acquired value for the presence or availability of labeled CG that were tagged with the ODN in the maternal blood sample can be acquired by amplification of the DNA molecules starting from the tagged CG sites using the ODN-directed and partially ligation mediated (LM-PCR) polymerase chain reaction. The skilled person will be well aware of suitable methods for ligating adaptor sequences to the DNA fragments. In LM-PCR of the present invention, an adaptor nucleic acid sequences are added onto both ends of each DNA fragments through preferably sticky end or blunt-end ligation, wherein each strand of an adaptor sequences is capable of hybridizing with a primer for PCR, thereby amplifying the DNA fragments to which the linkers have been ligated. In this aspect of the present invention, only one strand of the ligated partially complementary double-stranded adaptor sequence is used to anchor a primer for amplification of the labeled template DNA strand as shown in FIG. 8b. The second primer binds to the ODN sequence through complementary base pairing without contacts to the template DNA. The externally added primer should be at least 10 nucleotides and preferably at least 15 nucleotides in order to allow for a section of a primer to be involved in base pairing with the ODN without the complementary base pairing with the template DNA. This results in amplification of the labeled strands of nucleic acid samples, but not the original DNA fragment to which the adaptor sequences were ligated. In a preferred embodiment, the values of the amplified sequences are determined through real time quantitative polymerase chain reaction using oligonucleotide primers annealing within the regions shown in Tables 4, 5, 6 or 7 in the close vicinity to the labeled CGs as shown in FIG. 8a. Methods of qPCR are well known in the art. Representative, non-limiting conditions for qPCR are given in the Examples.

Yet, alternatively, the values of the amplified sequences, or DLRs, are determined through massive parallel sequencing. In this aspect of the embodiments, one strand of the ligated double-stranded adaptor sequence is used to anchor a primer for amplification of the labeled template DNA strand as shown in FIG. 8b. The second primer binds to the ODN sequence through complementary base pairing without contacts to the template DNA. Following PCR amplification, the values of the amplified sequences are determined through sequencing. This is only one exemplification of the presently described strategy for estimation of labeled nucleic acid through sequencing. In yet another aspect, the sub-fraction of the derivatized maternal sample DNA is selectively enriched through targeted PCR amplification prior to sequencing. Such PCR amplification makes use one primer bound complementary to the ODN or a part of it in the presence (5-7 nucleotide complementarity right at the target sites) or absence of complementarity to the template DNA, and the second primer bound through non-covalent complementary base pairing to the template DNA in the chromosomal regions shown in Tables 4-7.

In another embodiment of the invention, the experimentally acquired value for the presence or availability of labeled CG is estimated through qPCR, in a maternal blood sample that has not been subjected to adaptor ligation or pre-amplification, as shown in FIG. 8c. In this aspect, one primer to be used in qPCR hybridizes complementarily to the ODN altogether with 5-7 nucleotides of genomic template DNA near the derivatized CG site as described above and the second primer binds within the genomic DNA positions listed in Tables 4-7.

IV. Differentially Labeled Regions (DLRs).

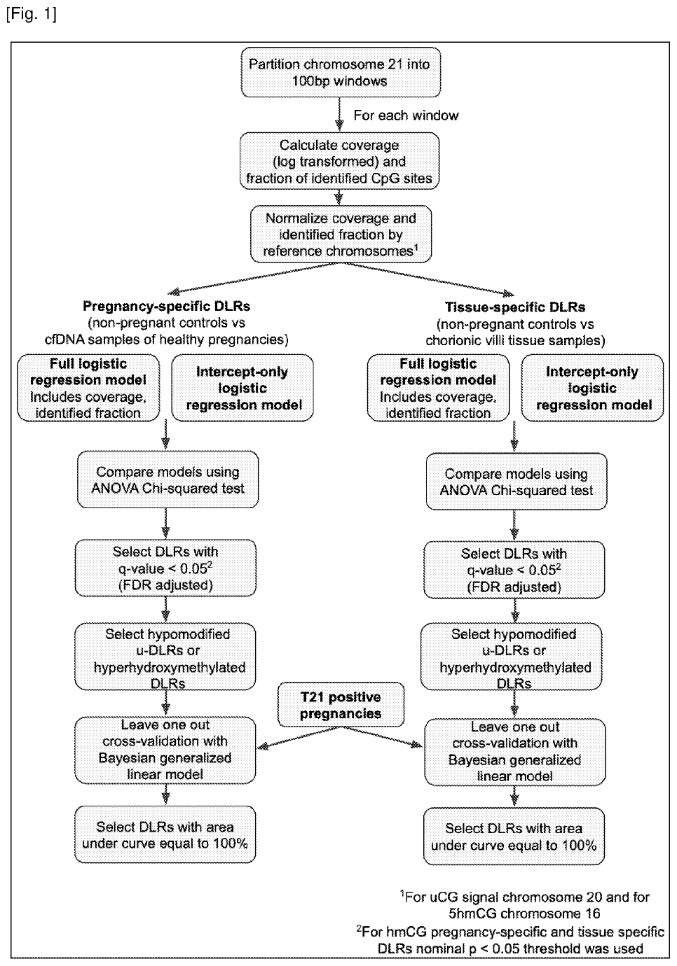

The diagnostic method of the invention employs a plurality of regions of chromosomal DNA wherein the regions are more intensively labeled in fetal DNA as compared to female peripheral blood samples. In theory, any chromosomic region with the above characteristics can be used in the instant diagnostic method. In particular, methods for identifying such DLRs are described in detail below and in the Examples (see Examples 1 and 2). Moreover, a large panel of DLRs for chromosomes 21, 13 and 18 suitable for use in the diagnostic methods, has now been identified (the strategy for identification of DLRs is shown in FIG. 1).

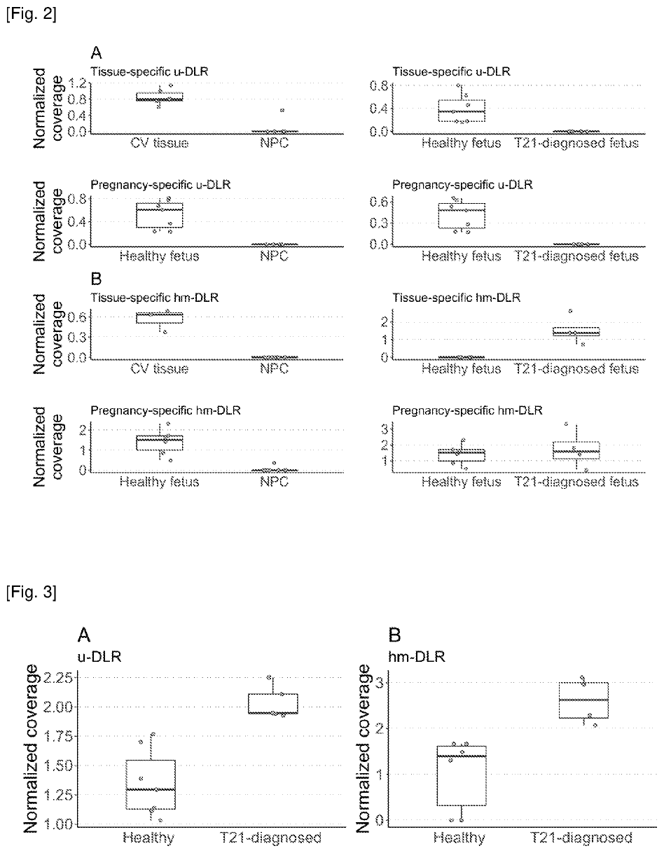

Furthermore, representative examples of a subset of these DLRs (4175 tissue-specific u-DLRs; 163 pregnancy-specific u-DLRs; 8815 tissue-specific hm-DLRs, 679 pregnancy-specific hm-DLRs) have been used to accurately predict trisomy 21, in a method based on analysis of fetal-specific DLRs in chromosome 21 by sequencing of labeled CG sites in a maternal blood sample. We also evaluated labeling differences between maternal blood samples of healthy and T21 positive pregnancies and identified 3,490 u-DLRs and 2,002 hm-DLRs which are shown in Tables 4 and 5, respectively. The effectiveness of the disclosed regions and methodologies for diagnosing fetal aneuploidy T21 has been demonstrated in FIGS. 2 and 3. Such DLRs are shown in the lists of Tables 4 and 5, which provide the selected DLRs for chromosome 21.

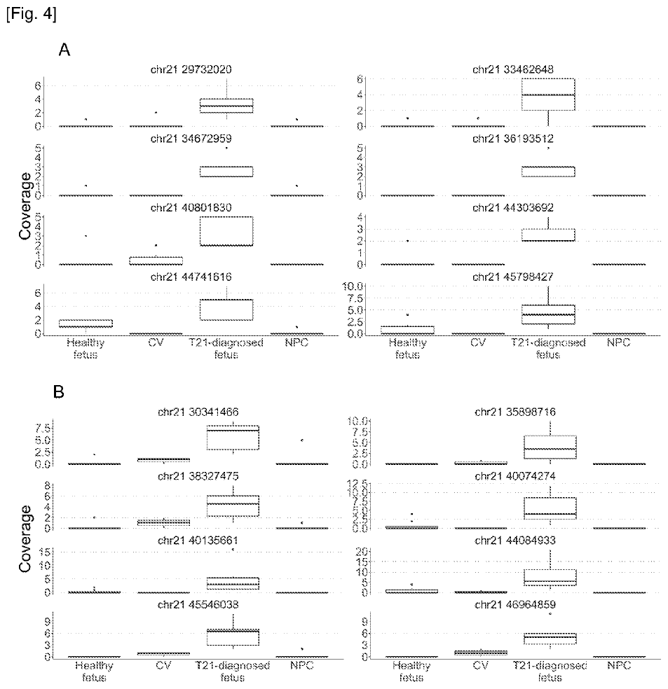

According to the second exemplary embodiment, DLRs restricted to individual CGs (CG-DLRs) have been identified in chromosomes 21 and X. Representative examples of a subset of these DLRs have been used to accurately predict trisomy 21, in a method based on analysis of fetal-specific hypomethylated or hyper-hydroxymethylated CG-DLRs in chromosome 21 by sequencing of labeled CG sites in a sample of maternal blood. Also, representative examples of a subset of these CG-DLRs have been used to accurately predict fetal gender, in a method based on analysis of fetal-specific CG-DLRs in chromosome X by sequencing of labeled CG sites in a sample of maternal blood. The effectiveness of the disclosed DLRs and methodologies for determination T21 aneuploidy and fetal gender has been demonstrated in FIG. 4 and FIG. 5. The list of DLRs is shown in Table 6.

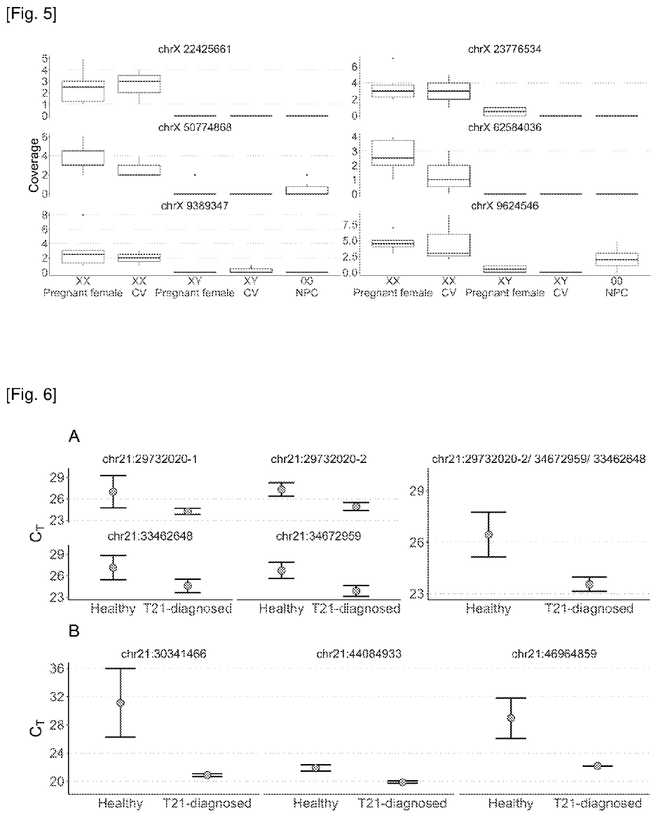

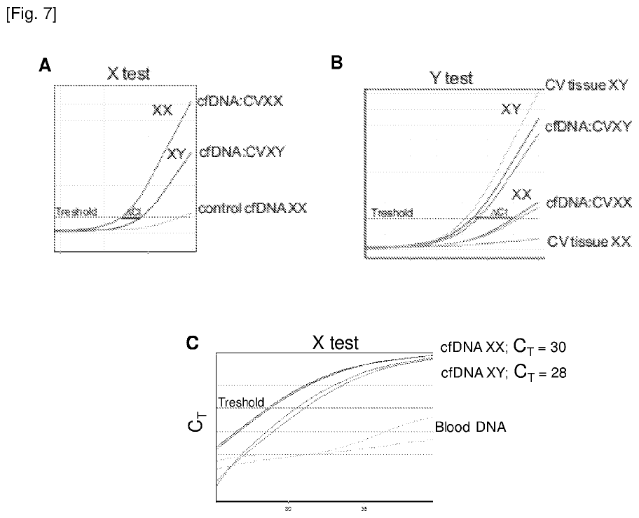

In the third exemplary embodiment, representative examples of a subset of the CG-DLRs have been used to accurately predict trisomy 21 and fetal gender, in a method based on analysis of fetal-specific DLRs in chromosome 21 and chromosome X and/or Y in a sample of maternal blood by qPCR. Thus, the effectiveness of the disclosed regions and methodologies for diagnosing trisomy 21 and fetal gender has been demonstrated in FIG. 6 and FIG. 7.

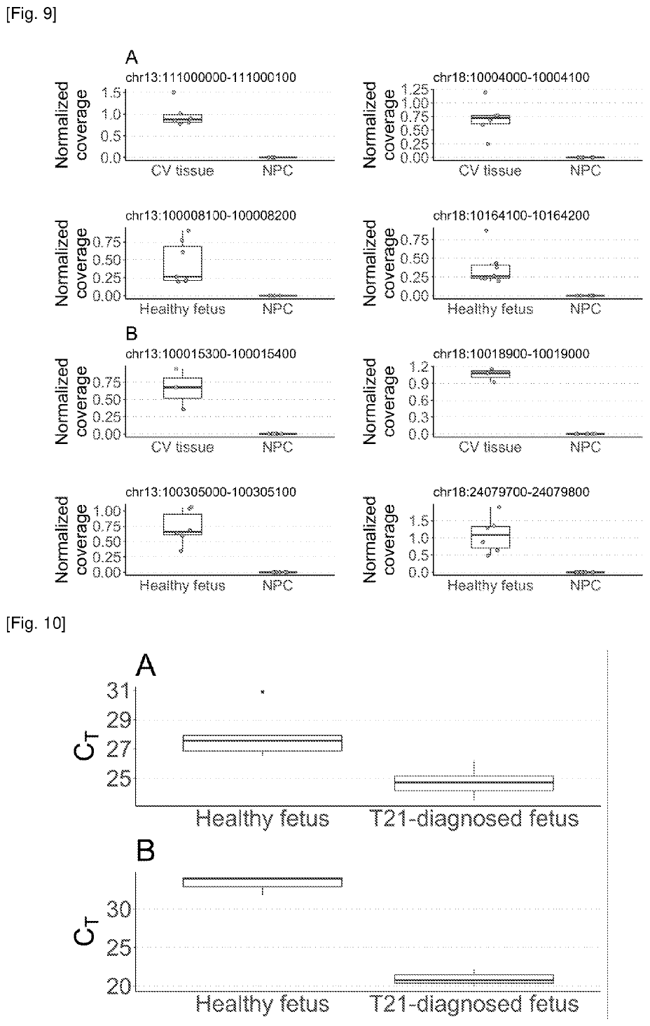

In other methods for detecting a fetal aneuploidy, the plurality of DLRs may be on chromosome 13, chromosome 18, to allow for diagnosis of aneuploidies of any of these chromosomes. In theory, any DMR with the above characteristics in a chromosome of interest can be used in the instant diagnostic method. Methods for identifying such DLRs in chromosome 13 and chromosome 18 are described in Example 1 and the effectiveness of the disclosed regions has been demonstrated in FIG. 9. The lists of selected DLRs for chromosomes 13 and 18 are provided in Table 7.

As used herein, the term “a plurality of DLRs” is intended to mean one or more regions or DLRs, selected from the list shown in Table 4-7. In various embodiments, the levels of the plurality of DLRs are determined for at least one region. Control regions or control DLRs also can be used in the diagnostic methods of the invention as a reference for evaluation of the labeled signal in the DLR region(s) of interest.

In a particularly preferred embodiment, the plurality of DLRs on chromosome 21 comprise one region or a combination of at least two regions, selected from the group shown in Table 6.

The invention also pertains to a composition comprising nucleic acid probes that selectively detect DLRs shown in Table 6.

The actual nucleotide sequence of any of the DLRs shown in Tables 4-7 is obtainable from the information provided herein together with other information known in the art. More specifically, each of the DLRs shown in Tables 4-7 is defined by a start base position on a particular chromosome, such as, for example “position 10774500” of chromosome 21. Furthermore, primers for targeted detection and/or amplification of a DLR can then be designed, using standard molecular biology methods, based on the nucleotide sequence of the DLR.

In another aspect, the invention provides nucleic acid compositions that can be used in the methods and kits of the invention. These nucleic acid compositions are informative for detecting DLRs. As described in detail in Example 3, at least one CG-DLR shown in Table 6 has been selected and identified as being sufficient to accurately diagnose trisomy 21 in a maternal blood sample during pregnancy of a woman bearing a trisomy 21 fetus.

V. Determining Levels of DLRs.

Labeling levels of the identified DLRs can be measured by sequencing or by qPCR.

Labeling levels of a plurality of regions as described above are determined in the unmethylated or hydroxymethylated DNA sample, to thereby obtain a labeling value for the DNA sample. As used herein, the term “the levels of the plurality of DLRs are determined” is intended to mean that the prevalence of the DLRs is determined. The basis for this is that in a fetus with a fetal trisomy 21 there will be a larger amount of the DLRs as a result of the trisomy 21, as compared to a normal fetus. In another aspect, when the T21-specific DLR are being used, the amount of such DLRs can be larger or lesser then the amount in a fetus without a fetal trisomy 21.

In a preferred embodiment, the levels of the plurality of DLRs are determined by real time quantitative polymerase chain reaction (qPCR), a technique well-established in the art. The term “the labeling value” is intended to encompass any quantitative representation of the level of DLRs in the sample. For example, the data obtained from qPCR can be used as “the labeling value” or it can be normalized based on various controls and statistical analyses to obtain one or more numerical values that represent the level of each of the plurality of DLRs in the testing DNA sample. The procedure for detection of DLRs by qPCR including primers' sequences, and the cycle conditions used were as described in Example 3.

In analysis of labeling intensity of DLRs by sequencing, the level of differential labeling was calculated for non-overlapping 100 bp regions. In more detail, for each window we computed the total log-transformed coverage and the fraction of identified CGs which we then normalized by the total log-transformed coverage and the fraction of identified CGs in reference chromosomes 16 (for uCG signal) and 20 (for hmC signal). For each window a full and null logistic regression models were fitted. Full model included coverage, identified fraction, and, for T21-specific DMRs, fetal sex and fetal fraction, as independent variables. Coverage and identified fraction were excluded from the null model. ANOVA Chi-squared test was used to compare full and null models to obtain p value. In cases where models did not converge fetal sex was removed and p value evaluated again. Model statistics were moderated using empirical Bayes. FDR was used to adjust p values for multiple testing and q<0.05 was used as significance threshold.

For each pregnancy-specific or tissue-specific DLR a leave-one-out cross-validation procedure was performed in order to determine its ability to diagnose T21. For each cross-validation cycle Bayesian generalized linear model (Gelman et al. 2008) with normalized coverage and identified CG as independent variables was constructed on the training samples. The model was then applied on the testing sample returning the predicted probability of the sample belonging to the T21 category. After all the cross-validation cycles the prediction probabilities for all samples were taken together. Various thresholds that would determine the discrete sample class from continuous probability measurement may have different effects on predictor's specificity and sensitivity. Therefore, a receiver-operating characteristic curve analysis was performed to estimate the effect of any threshold. The area under receiver-operating characteristic curve (AUC) indicates the overall accuracy of the model. Those DLRs for which the area under the curve was equal to 100% and, therefore, could achieve 100% prediction accuracy, were deemed to be the T21-predictive DLRs.

An approach that would combine individual DLRs into a single predictive model is also possible. Such model could be one of but not limited to elastic net, random forest or support vector machine. Model would be evaluated in the same way by assessing receiver-operating characteristic and using cross-validation for parameter tuning. Also, bootstrap could be used instead of cross-validation. Other model accuracy measures could be employed, and data could be transformed in different ways. Interactions of DLRs could be taken into account to build new composite features that would be used for subsequent model training and evaluation.

VI. Comparison to a Standardized Reference Value.

The labeling value of the fetal DNA (also referred to herein as the “test value”) present in the maternal peripheral blood is compared to a standardized reference value, and the diagnosis of trisomy 21 (or lack of such fetal trisomy 21) is made based on this comparison. Typically, the test value for the fetal DNA sample is compared to a standardized normal reference value for a normal fetus, and diagnosis of fetal trisomy 21 is made when the test value is higher than the standardized normal reference labeling value for a normal fetus. In another aspect, the test value can be lower than the standardized normal reference labeling value for a normal fetus.

Alternatively, the test value for the labeled DNA sample can be compared to a standardized reference labeling value for a fetal trisomy 21 fetus, and diagnosis of fetal trisomy 21 can be made when the test value is comparable to the standardized reference labeling value for a fetal trisomy 21 fetus.

To establish the standardized normal reference labeling values for a normal fetus, maternal blood samples from the pregnant women carrying a normal fetus are subjected to the same steps of the diagnostic method, namely amplification of the ODN-derivatized CGs and their neighboring genomic sequences to obtain a reference DNA sample, and then determining the labeling value and the levels of at least one region of chromosomal DNA by sequencing or qPCR wherein selected from Tables 4-7.

In order to establish the standardized normal reference methylation values for a normal fetus, healthy pregnant women carrying healthy fetuses or healthy non-pregnant women are selected. Pregnant women are of similar gestational age, which is within the appropriate time period of pregnancy for screening fetal chromosomal aneuploidy, typically within the first trimester of pregnancy. Standardized reference labeling values for a T21 fetus can be established using the same approach as described above for establishing the standardized reference values for a healthy fetus, except that the maternal blood samples used to establish the T21-specific reference values are from pregnant women who have been determined to be carrying a fetus with fetal trisomy 21.

### EXAMPLES

### Example 1. Identification of DLRs

This example provides the methodology for the preparation of the labeled genomic libraries of the mentioned-above biological samples for genomic mapping of unmodified or hydroxymethylated CGs. Also, this example provides the strategy for DLRs determination and how DLRs for detection of trisomy T21 were preferentially chosen. FIG. 8b shows the application of the sequencing methodology for the identification of DLRs. In this example, DLRs in chromosomes 13 and 18 were also identified.

Biological Samples.

We performed analysis of three distinct sample types, enabling a characterization of the unmethylated and hydroxymethylated CGs in DNA obtained from plasma of pregnant women; we created single CG resolution uCG and 5hmCG maps of placental chorionic villi (CV) tissue samples from the 1st trimester abortions (CVS; n=6 of uCG and n=3 of 5hmCG); cfDNA samples of female non-pregnant controls (NPC; uCG n=6 and 5hmCG n=7) and cfDNA samples of pregnant women carrying healthy fetuses (uCG n=7 and 5hmCG n=6) or fetuses with the trisomy 21 (uCG n=5 and 5hmCG n=4).

Circulating DNA from maternal blood samples was extracted using the MagMax Nucleic Acid Extraction kit (Thermo Fisher Scientific (TS)) or the QIAamp DNA blood Midi Kit (QIAGEN), and DNA from chorionic villi tissue was prepared by phenol extraction.

All the maternal peripheral blood DNA samples (1st trimester pregnancies) and chorionic villi samples (1st trimester abortions) were obtained at Tartu University Hospital (Tartu, Estonia) through collaboration with Tartu University (Estonia). Consent forms approved by the Research Ethics Committee of the University of Tartu (ethical permission No. 246/T-21 and 213/T-21) were collected for each of the mother participated.

Mapping of Unmodified/Hydroxymethylated CGs in DNA Extracted from Biological Samples.

In uTOP-seq, 4-10 ng of cfDNA (or 100 ng of CV tissue DNA, sheared to 200 bp by Covaris sonicator) were labeled with 0.11 ΣM eM.Sssl (Kriukienė et al. 2013) in 10 mM Tris-HCl (pH 7.4), 50 mM NaCl, 0.5 mM EDTA buffer supplemented with 200 μM Ado-6-azide cofactor (Masevicius et al, 2016) for 1 h at 30° C. followed by thermal inactivation at 65° C. for 20 min and Proteinase K treatment (0.2 mg/ml) for 30 min at 55° C. and finally column purified (GeneJET PCR purification kit, (TS)). In hmTOP-seq, 5hmC glycosylation was carried with 5-10 ng of cfDNA supplemented with 50 μM UDP-6-azide-glucose (Jena Bioscience) and 2.5-5 U T4 β-glucosyltransferase (TS) for 1 h 37° C. followed by enzyme inactivation at 65° C. for 20 min and column purification (GeneJET PCR Purification kit (TS)). After ligation of the partially complementary adapters as described previously (Staševskij et al. 2017), covalently labeled DNA was supplemented with 20 μM alkyne-containing DNA oligonucleotide (which was biotinylated for construction of 5hmC maps) (ODN; 5′-T(alkyneT)TTTTGTGTGGTTTGGAGACTGACTACCAGATGTAACA-3′ (or -(biotin)-3′), Base-click) and 8 mM CuBr: 24 mM THPTA mixture (Sigma) in 50% of DMSO, incubated for 20 min at 45° C. and subsequently diluted to <1.5% DMSO before a column purification (GeneJET NGS Cleanup Kit, Protocol A (TS)). DNA recovered after biotinylation step was incubated with 0.1 mg Dynabeads MyOne Cl Streptavidin (TS) in a buffer A (10 mM Tris-HCl (pH 8.5), 1 M NaCl) at room temperature for 3 h on a roller. DNA-bound beads were washed 2× with buffer B (10 mM Tris-HCl (pH 8.5), 3 M NaCl, 0.05% Tween 20); 2× with buffer A (supplemented with 0.05% Tween 20); 1× with 100 mM NaCl and finally resuspended in water and heated for 5 min at 95° C. to recover enriched DNA fraction. Purified DNA after oligonucleotide conjugation (uCG) or biotin-enrichment (5hmC) was subsequently used in a priming reaction with 1 U Pfu DNA polymerase (TS), 0.2 mM dNTP, 0.5 μM complementary priming oligonucleotide (EP; 5′-TGTTACATCTGGTAGTCAGTCTCCAAACCACACAA-3). The reaction mixture was incubated at the following cycling conditions: 95° C. 2 min; 5 cycles at 95° C. 1 min, 65° C. 10 min, 72° C. 10 min. Amplification of a primed DNA library was carried out by adding the above reaction mixture to 100 μl of amplification reaction containing 50 μl of 2× Platinum SuperFi PCR Master Mix (TS) and barcoded fusion PCR primers A(Ad)-EP-barcode-primer (63 nt) and trP1(Ad)-A2-primer (45 nt) at 0.5 μM each. Thermocycler conditions: 94° C. 4 min; 15 cycles (uCG) or 17 cycles (5hmC) at 95° C. 1 min, 60° C. 1 min, 72° C. 1 min. The final libraries were size-selected for −270 bp fragments (MagJET NGS Cleanup and Size Selection Kit, (TS)), and their quality and quantity were tested on 2100 Bioanalyzer (Agilent). Libraries were subjected to Ion Proton (TS) sequencing.

Data Analysis.

Raw TOP-seq and hmTOP-seq sequencing reads were processed as described in Staševskij et al. (2017) and Gibas et al. (2020, accepted) except for the 3′ sequence ends where adapter sequences were trimmed only if they were identified using cutadapt with maximum allowed error rate 0.1 (Martin 2011). Processed reads were mapped to reference human genome version hg19 and coverage for each CG dinucleotide was computed as the total number of reads starting at or around the CG dinucleotide on either of its strands. We define CG coverage as the total number of reads, c, on any strand starting within absolute distance, d. We retained only reads with d≤3. Only reads aligned to chromosomes 1 to 22, X and Y were used for further analysis. On average, 40% of the raw reads were retained for downstream analysis per sample.

Outlier identification was performed separately for uCG and 5hmC samples. CG coverage matrices were transformed using Hellinger transformation (Legendre and Gallagher, 2001) and then represented in two-dimensional space using non-metric multidimensional scaling (nMDS) with Bray-Curtis similarity index (Bray and Curtis, 1957). Samples that were further than two standard deviations away from the mean of their own sample group (cfDNA of non-pregnant controls, other cfDNA, CV tissue) in either nMDS1 or nMDS2 dimension were deemed outliers and removed from further analysis. There were three outlying samples in uCG and one in 5hmCG dataset.

Identification of DLRs in Chromosomes 21, 13 and 18.

The strategy for DLR identification is show in FIG. 1. We partitioned the chromosome 21 or 13 or 18 into 100 bp-wide non-overlapping windows. For each window we computed the total log-transformed coverage and the fraction of CGs covered which we then normalized by the total log-transformed coverage and the fraction of identified CGs in reference chromosomes 16 (for uCG) and 20 (for hmC).

First, we obtained the pregnancy-specific u-DLRs by comparing NPC samples with cfDNA samples of healthy pregnancies. For each window a full and null logistic regression models were fitted. Full model included coverage, identified fraction, and, for T21-specific DLRs, fetal sex and fetal fraction, as independent variables. Coverage and identified fraction were excluded from the null model. ANOVA Chi-squared test was used to compare full and null models to obtain p value. In cases where models did not converge fetal sex was removed and p value evaluated again. Model statistics were moderated using empirical Bayes adjustment. FDR was used to adjust p values for multiple testing and q<0.05 was used as significance threshold.

Next, we used the same strategy to obtain tissue-specific u-DLRs (FDR q<0.05; logistic regression) by comparing NPC and CV tissue samples. The same analytic approach was used separately for uCG and hmCG data. In case of hm-DLRs, nominal p value threshold was used when analysis did not yield any FDR significant DLRs.

Further, for each hypomodified pregnancy-specific and tissue-specific u-DLR or hyper-hydroxymethylated pregnancy-specific and tissue-specific hm-DLR in chromosome 21 a leave-one-out cross-validation procedure was performed in order to determine its ability to diagnose T21. For each cross-validation cycle Bayesian generalized linear model (Gelman et al. 2008) with normalized coverage and identified CG as independent variables was constructed on the training samples. The model was then applied on the testing sample returning the predicted probability of the sample belonging to the T21 category. After all the cross-validation cycles the prediction probabilities for all samples were taken together. Various thresholds that would determine the discrete sample class from continuous probability measurement may have different effects on predictor's specificity and sensitivity. Therefore, a receiver-operating characteristic curve analysis was performed to estimate the effect of any threshold. The area under receiver-operating characteristic curve indicates the overall accuracy of the model. Those DLRs for which area under the curve was equal to 100% and, therefore, could achieve 100% prediction accuracy, were deemed to be T21-predictive DLRs (FIG. 1).

Using the strategy for DLR determination in chromosome 21, we obtained 2,761 pregnancy-specific u-DLRs (FDR q<0.05) and 16,555 fetal tissue-specific u-DLRs (FDR q<0.05; logistic regression). For hm-DLR identification, we used nominal p<0.05 threshold and identified 4,930 pregnancy-specific hm-DLRs and 15,986 tissue-specific hm-DLRs.

An in-depth investigation of the identified DLRs between non-pregnant female peripheral blood and placental DNA samples or non-pregnant and pregnant female cfDNA samples, has led to the selection of a list of DLRs located on chromosome 21 for diagnosing trisomy 21. The selection criteria of the regions were based firstly on the labeling intensity status of the regions in maternal blood samples and CV DNA samples, or on the labeling intensity status of the regions in the non-pregnant and pregnant female maternal blood samples. More specifically, the selected regions should demonstrate a high labeling intensity status in CV tissue DNA and a low labeling intensity or absence of labeling in peripheral blood samples of NPCs, or should show a high labeling intensity status in pregnant female blood samples and a low labeling intensity or absence of labeling in NPCs. Using leave-one-out cross-validation as described above we discovered 4175 tissue-specific u-DLRs; 163 pregnancy-specific u-DLRs; 8815 tissue-specific hm-DLRs, 679 pregnancy-specific hm-DLRs in chromosome 21 that classified the samples according to fetal karyotype with 100% accuracy (the selected DLRs are shown in Tables 4 and 5, for the uCG and hmCG signal, respectively) (FIG. 2).

Furthermore, considering global epigenetic changes in Down syndrome affected fetuses (Jin et al. 2013), we also employed an alternative approach to identify the trisomy 21-specific DLRs. We evaluated modification differences between cfDNA samples of healthy and T21-diagnosed pregnancies and identified differentially modified DLRs. A logistic regression model was fitted to each 100 bp window with the CG-coverage and CG-fraction as independent variables and karyotype as the response variable, as above. In addition, we adjusted for possible confounding effects of fetal fraction and fetal gender which could not be accounted for in the previous analyses. With such approach, we identified 3,490 u-DLRs and 2,002 hm-DLRs (FDR q<0.05; logistic regression). The selected T21-specific DLRs that discriminate most the sample groups of healthy and T21-diagnosed pregnancies are shown in Tables 4 and 5, for uCG and hmCG signal, respectively) (FIG. 3).

Using the same strategy for DLR identification shown in FIG. 1 we also identified DLRs in chromosomes 13 and 18. For chromosome 13, we obtained 1,394 pregnancy-specific u-DLRs (FDR q<0.05) and 25,091 fetal tissue-specific u-DLRs (FDR q<0.05; logistic regression) and using nominal p<0.05 threshold 4,255 pregnancy-specific hm-DLRs and 22,526 tissue-specific hm-DLRs. For chromosome 18, we obtained 1,321 pregnancy-specific u-DLRs (FDR q<0.05), 22,121 fetal tissue-specific u-DLRs (FDR q<0.05; logistic regression) and 3,626 pregnancy-specific hm-DLRs and 20,780 tissue-specific hm-DLRs. The lists of the selected DLRs across chromosomes 13 and 18 are shown in Table 7 (FIG. 9).

The total number of fetal specific hypomethylated and hyper-hydroxymethylated tissue- and pregnancy-specific DLRs identified across chromosomes 21, 13 and 18 is summarized in Table 1.

TABLE 1
Numbers of pregnancy- and tissue-specific DLRs
identified across chromosomes 21, 13 and 18.

| | No. of | No. of | No. of hyper- | No. of hyper- |
|---|---|---|---|---|
| | hypo- | hypo- | hydroxy- | hydroxy- |
| | methylated | methylated | methylated | methylated |
| | tissue- | pregnancy- | tissue- | pregnancy- |
| | specific | specific | specific | specific |
| Chromosome | u-DLRs | u-DLRs | hm-DLRs | hm-DLRs |
| Chr21 | 4175 | 163 | 8815 | 679 |
| Chr13 | 25091 | 1394 | 22526 | 4255 |
| Chr18 | 22121 | 1321 | 20780 | 3626 |

### Example 2. Identification of Individual Labeled CGs for Detection of Trisomy 21 and Fetal Sex

This example provides the strategy for determination of individual labeled CGs (CG-DLRs) following analysis of the samples described in Example 1 that can be used for detection of fetal trisomy T21.

An investigation of labeling intensities of uCGs and hmCGs in peripheral blood samples of women that were confirmed to be carrying a fetus with trisomy 21 against labeling intensities of uCGs and hmCGs in the three types of control samples, i.e. placental CV tissue DNA, peripheral blood samples of non-pregnant women and peripheral blood samples of women pregnant with healthy fetuses, has led to the selection of individual CG-DLRs located on chromosome 21 for detection of fetal T21. The selection criteria of the CG-DLRs were based firstly on a labeling intensity status of CGs in blood samples of women pregnant with T21-diagnosed fetuses. More specifically, the selected CG-DLRs should demonstrate a high labeling intensity status in blood samples of women pregnant with T21-diagnosed fetuses and a low labeling intensity or absence of labeling in the three other sample types: CV tissue DNA, peripheral blood samples of NPC and pregnant female carrying a healthy fetus.

The CGs with non-zero coverage and non-zero variance were used. The read coverage was log transformed. CGs from chromosome 21 were used for detection of T21 markers. Samples from non-pregnant female and pregnant with healthy fetuses women and CV tissue samples were marked as control, whereas only the female samples with T21 positive fetuses were marked as cases. A linear regression model was fitted for every CG, and resulting model fits were moderated using empirical Bayes adjustment. The CGs with FDR q value less than 0.05 and log fold change more than 1.2 were taken as significant. The list of the selected T21 CG-DLRs is shown in Table 6 (FIG. 4).

Identification of CG-DLRs for Determination of Fetal Sex.

Similarly, CGs from chromosome X (and Y) were analyzed for identification of CG-DLRs for fetal gender determination. A no intercept linear regression model was fitted for each CG and a contrast fit was used to determine differences between male and female samples. Resulting model fits were moderated using empirical Bayes adjustment. The CGs with FDR q value less than 0.05 and log fold change more than 1 were taken as significant. The list of the selected gender CG-DLRs is shown in Table 6 (FIG. 5).

### Example 3. Evaluation of CG-DLRs by qPCR

In this example, individual CGs or CG-DLRs identified according to the methodology described in Examples 1 and 2 were used for their validation by qPCR. A flowchart diagram of the methodology is shown in FIGS. 8a and c. Several experiments were carried out to analyze and validate the identified DLRs or individual CGs. These experiments include an evaluation of the variability and reproducibility of the labeling intensity among different individuals and among technical replicates.

Detection of Fetal Trisomy T21 by qPCR.

The difference in labeling intensity at specific CG-DLRs, shown in Table 6, was tested in blood samples of pregnant female carrying healthy or T21-diagnosed fetuses (FIG. 6). Briefly, DNA of maternal blood sample was treated as described in Example 1. Then, 0.5 ng of the final amplified DNA were used for measurement of the labeling intensity of u-CG-DLRs and hm-CG-DLRs by qPCR with a Rotor-Gene 0 real-time PCR system (Qiagen) using Maxima SybrGreen/ROX qPCR Master Mix (TS). 0.3 mM of each primer pair used in each reaction, wherein one of the primers binds complementarily to a genomic region in close proximity to the CG site (its 5′ end anneals more than 5 nucleotides to the CG being analyzed), and another primer binds in a vicinity of the CG to allow PCR amplification of the region (or selected DLR) to occur. The amplification conditions were set as: 95° C. for 10 min, 40 cycles 95° C. for 15 s, 60° C. for 60 s.

In this embodiment, the plurality of CG-DLRs on chromosome 21 comprises one region or a combination of at least two regions, selected from Table 6. The invention also pertains to a composition comprising nucleic acid probes that selectively detect the regions shown in Table 6, preferably, the pair/set of oligonucleotide primers are selected from Table 2.

TABLE 2
[First position of the genomic coordinates
of the selected u-CG-DLRs
and hm-CG-DLR on chromosome 21 and nucleotide
sequences of the primers for
determination of fetal trisomy T21 by qPCR.]

| | PCR | |
|---|---|---|
| u-CG-DLR | product, | |
| coordinate | length | Primer sequence |
| Chr21: | 29732020-1, | Seq ID 1: |
| 29732020 | 109 bp | 5′CAACTCCCTACAG |
| | | CCCCTTG |
| | | Seq ID 2: |
| | | 5′AAATTGCATGATT |
| | | CCCCTGACA |
| Chr21: | 29732020-2, | Seq ID 3: |
| 29732020 | 67 bp | 5′ATGACTGGCTTATTTC |
| | | ACTTAGCATC |
| | | Seq ID 4: |
| | | 5′AGTCCTGCTATATGCA |
| | | ACACCTT |
| Chr21: | 33462648, | Seq ID 5: |
| 33462648 | 97 bp | 5′GGTATTTACAAAAGT |
| | | CTGCACCTTAGTC |
| | | Seq ID 6: |
| | | 5′CTGCCAACTTCACCC |
| | | AGAGT |
| Chr21: | 34672959, | Seq ID 7: |
| 34672959 | 73 bp | 5′TAGAAATCTTTAGGA |
| | | GGTGGTGAATG |
| | | Seq ID 8: |
| | | 5′CATGGTGGAAGAGAT |
| | | GGGC |
| | PCR | |
| hm-CG-DLR | product, | |
| coordinate | length | Primer sequence |
| Chr21: | 30341466, | Seq ID 9: |
| 30341466 | 101 bp | 5′GCAGAGGTTGCAG |
| | | TGAGCTG |
| | | Seq ID 10: |
| | | 5′GTCTGGATGCAAAA |
| | | ATCCCTTT |
| Chr21: | 46964859, | Seq ID 11: |
| 46964859 | 88 bp | 5′GCTGTCCCTGTGGT |
| | | TAAGGTC |
| | | Seq ID 12: |
| | | 5′GCCACCACAACAGC |
| | | ACCA |
| Chr21: | 44084933, | Seq ID 13: |
| 44084933 | 89 bp | 5′CCCCATCACCAACT |
| | | TCACTC |
| | | Seq ID 14: |
| | | 5′GAAACTGAGTCTC |
| | | TCGCAAGG |

Detection of Fetal Gender by qPCR.

In another embodiment of the invention, the experimentally acquired value for the presence or availability of labeled CGs is estimated through qPCR, in a total untreated, i.e. non-ligated to adaptors and non-preamplified, maternal blood sample as shown in FIG. 8c, for fetal gender determination. Notably, analysis of the selected CG-DLRs in chromosome X is sufficient for detection of fetal gender. This is only one exemplification of the strategy; the similar strategy may be used for determination of fetal trisomy.

Firstly, the difference in the abundance of DLR regions starting at specific CGs shown in Table 6 was tested in the 1st trimester CV tissue DNA of both genders and non-pregnant female blood sample DNA. Then, we mixed CV tissue DNA and non-pregnant female peripheral blood plasma DNA to the ratios 20/80 and 0/100 of the CV and plasma DNA, respectively. 10 ng of each sample mixture were labeled and derivatized with the ODN as described above. Next, 1.5 ng of each sample was analyzed in replicates by qPCR. The coordinates of the u-CG-DLRs on chromosomes X and Y and primers for qPCR are shown in Table 3.

TABLE 2=3
[First position of the genomic coordinates
of the selected u-CG-DLRs on chromosomes
X and Y and nucleotide sequences of the
primers for determination of
fetal gender by qPCR.]

| | PCR | |
|---|---|---|
| u-CG-DLR | product | |
| coordinate | length | Primer sequence |
| ChrX: | 160 bp | Seq ID 15: 5′-CCTCTCTATGGGCAGT |
| 138802516 | | CGGTGATTGACCTGCTTCCTGTGTTGAGC |
| | | Seq ID 16: 5′-TGTTACATCTGGTAGT |
| | | CAGTCTCCAAACCACACAAAAAAGTGGAG |
| ChrY: | 123 bp | Seq ID 17: 5′-GTAGAAAAAAGTAGA |
| 14774154 | | AACAGCAAGGGGAAG |
| | | Seq ID 18:5-TGTTACATCTGGTAGTC |
| | | AGTCTCCAAACCACACAAAAAAGCCCCT |

In more detail, DNA of each sample were labeled with eM.Sssl MTase in the presence of 200 μM Ado-6-azide cofactor for 1 hour at 30° C. as described in Example 1 followed by column purification (Oligo Clean&Concentrator-5, Zymo Research). Then, DNA eluted in 8 ul of Elution Buffer was supplemented with 20 uM alkyne DNA oligonucleotide (ODN, 5′-T(alkyneU)TTTTGTGTGGTTTGGAGACTGACTACCAGATGTAACA), the mixture of 8 mM CuBr and 24 mM of THPTA (Sigma) in 50% of DMSO, incubated for 20 min at 45° C. and subsequently diluted to <1.5% DMSO before purification through the GeneJET NGS Cleanup kit (TS). 1.5 ng of the purified DNA were used for measurement of the labeling intensity of uCGs by qPCR with a Rotor-GeneQ real-time PCR system (Qiagen) using Maxima SybrGreen/ROX qPCR Master Mix (TS). 0.3 mM of each primer pair was used in each reaction, wherein one of the primers binds complementarily to the ODN and to 5 nucleotides of the template genomic DNA adjacent to the derivatized CG site, and another primer binds in a vicinity of the CG to allow PCR amplification of the region (or selected DLR) to occur. The amplification program was set as: 95° C. for 10 min, 40 cycles 95° C. for 15 s, 65° C. for 30 s, 72° C. for 30 s (FIG. 7a,b,c).

### Example 4. qPCR-Based Noninvasive Diagnostics of Trisomy 21

This example describes the independent validation of non-invasive testing for fetal trisomy 21. For this purpose, we have performed qPCR-based analysis of a small group of samples which have not been used in the previous Examples for identification of validation of DLRs. The group consists of 3 maternal peripheral blood samples from women bearing a normal fetus and 2 maternal peripheral blood samples from women bearing a trisomy 21-affected fetus.

These maternal peripheral blood samples were obtained at a gestational age of between 12-13 weeks at Tartu University Hospital (Tartu, Estonia) through collaboration with Tartu University (Estonia). Consent forms approved by the Research Ethics Committee of the University of Tartu (ethical permission No. 246/T-21 and 213/T-21) were collected for each of the mother participated.

The fetal specific approach used herein is illustrated schematically in FIG. 8a, wherein the ability to discriminate normal from trisomy 21 cases is achieved by comparing the values obtained from normal and trisomy 21 cases using T21-specific differentially modified CG dinucleotides, or CG-DLRs, located on chromosome 21. A fetus with trisomy 21 has a differentially modified genome in relation to normal genome and an extra copy of chromosome 21, and thus the increased abundance of a fetal specific region compared to a normal fetus. Therefore, the amount of T21-specific fetal region will increase more in fetuses with trisomy 21 compared to normal cases.

An in-depth investigation of our previously identified DLRs, described in Examples 1 and 2, has led to selection of DLRs located on chromosome 21. A group of selected DLRs has been used for identification of fetal trisomy 21 by qPCR (Example 3). These DLRs demonstrate a hypomethylated or hyper-hydroxymethylated, and thus more labeled, status in peripheral blood DNA of pregnant women carrying a T21-diagnosed fetus and a hypermethylated or hypo-hydroxymethylated, and thus less labeled, status in CV tissue DNA and peripheral blood DNA of pregnant women carrying a normal fetus and in peripheral blood DNA of non-pregnant women in order to achieve the enrichment of fetal T21-specific CG-labeled regions. These selected CG-DLRs shown in Table 2 were used for analysis of the samples by qPCR.

The procedure of sample processing and qPCR cycle conditions used were as described in Examples 1 and 3. Briefly, 5-10 ng of maternal cfDNA was covalently derivatized with the ODN and the adaptors were ligated to the ends of DNA fragments. The labeled CG regions were enriched through the ODN-mediated polymerization of the adjacent genomic regions and such regions were subsequently amplified using the primers complementary to the ODN and one strand of the adaptors. Then, the amounts of u-CG-DLRs and hm-CG-DLRs was calculated by qPCR as shown in Example 3 using a combination of CG-DLRs and qPCR primers listed in Table 2.

Comparing the obtained test values of the samples with known karyotype (the T21-diagnosed samples show lower test values than normal cases), all T21-diagnosed samples were confirmed as having trisomy 21, indicating 100% specificity and 100% sensitivity of the approach (FIG. 10).

### APPENDICES

TABLE 4
[The coordinate is shown for the first base pair of 100 bp u-DLRs in chromosome 21]
Pregnancy-specific u-DLRs

| | | | | | | | |
|---|---|---|---|---|---|---|---|
| 10774500 | 26212900 | 35812700 | 38891600 | 43228800 | 45323700 | 46743900 | 47331000 |
| 11025700 | 26835100 | 35819500 | 38946900 | 43470300 | 45330400 | 46751000 | 47331900 |
| 15169700 | 28041300 | 35879100 | 38969700 | 43519400 | 45355100 | 46808700 | 47362600 |
| 15770300 | 28074300 | 36073900 | 39202100 | 43708100 | 45392900 | 46812700 | 47390300 |
| 16130900 | 28759100 | 36089600 | 39507100 | 43714600 | 45400400 | 46837800 | 47419000 |
| 16577200 | 28942700 | 36220800 | 39544400 | 43728400 | 45597600 | 46847100 | 47451100 |
| 17308600 | 29288000 | 36437300 | 39690100 | 43782100 | 45734900 | 46934400 | 47479200 |
| 17333200 | 31008200 | 36478700 | 39891300 | 43864600 | 45748300 | 46946300 | 47498400 |
| 18086100 | 32374100 | 36701300 | 41001100 | 43864800 | 45753500 | 46973100 | 47502800 |
| 18676300 | 32639100 | 36917100 | 41292800 | 43876000 | 45790600 | 46995500 | 47536100 |
| 18940600 | 32915800 | 37085900 | 42099000 | 44061700 | 45842200 | 46997700 | 47542700 |
| 20437200 | 33522600 | 37192500 | 42127100 | 44113000 | 46036100 | 46999800 | 47549500 |
| 20608700 | 33533900 | 37218700 | 42212900 | 44191100 | 46182600 | 47057200 | 47559700 |
| 21354200 | 33591700 | 37352800 | 42424900 | 44196200 | 46312000 | 47181700 | 48047900 |
| 21670800 | 33954100 | 37493000 | 42595400 | 44208900 | 46359300 | 47211600 | 48079600 |
| 22564300 | 34369300 | 37527800 | 42694800 | 44346000 | 46396600 | 47212000 | 9901200 |
| 24387600 | 34406400 | 37970500 | 42732500 | 44474700 | 46415700 | 47213500 | |
| 24474800 | 34483300 | 38066600 | 42746400 | 44511200 | 46418600 | 47245100 | |
| 25233800 | 34851100 | 38092400 | 42928900 | 44754300 | 46545400 | 47273200 | |
| 25693500 | 35365900 | 38104700 | 42936000 | 45065800 | 46720900 | 47287600 | |
| 26152100 | 35531600 | 38385400 | 43112600 | 45156300 | 46738900 | 47315300 | |

Tissue-specific u-DLRs. Only 1000 selected DLRs are shown

| | | | | | | | |
|---|---|---|---|---|---|---|---|
| 10027900 | 15984000 | 17333200 | 18351000 | 19378400 | 20630400 | 21741100 | 22819200 |
| 10395200 | 15993500 | 17333300 | 18356700 | 19379400 | 20633700 | 21743700 | 22820400 |
| 10527800 | 16003800 | 17344200 | 18361400 | 19382400 | 20655700 | 21745700 | 22830600 |
| 10551600 | 16009000 | 17364800 | 18387200 | 19390200 | 20668400 | 21746900 | 22842800 |
| 10603000 | 16010900 | 17377300 | 18389400 | 19391100 | 20685000 | 21755000 | 22848600 |
| 10713200 | 16015300 | 17382300 | 18399000 | 19392100 | 20698000 | 21765500 | 22866100 |
| 10757400 | 16016600 | 17384200 | 18418800 | 19392900 | 20701500 | 21771800 | 22880300 |
| 10762300 | 16025200 | 17389700 | 18426600 | 19397200 | 20706300 | 21775600 | 22896500 |
| 10762500 | 16033800 | 17392100 | 18433700 | 19400200 | 20715200 | 21775700 | 22925400 |
| 10807400 | 16039700 | 17396700 | 18444200 | 19406900 | 20719900 | 21802300 | 22926000 |
| 10812800 | 16046800 | 17400700 | 18449500 | 19415500 | 20748600 | 21809600 | 22926600 |
| 10821600 | 16051800 | 17405400 | 18461000 | 19427200 | 20749700 | 21814100 | 22936200 |
| 10824800 | 16056200 | 17405500 | 18483000 | 19427900 | 20759800 | 21826100 | 22947400 |
| 10826000 | 16058300 | 17422500 | 18492200 | 19429700 | 20763100 | 21831900 | 22970300 |
| 10836600 | 16065200 | 17423000 | 18497900 | 19432000 | 20780900 | 21832100 | 22975500 |
| 10851100 | 16065400 | 17423200 | 18519100 | 19443000 | 20790400 | 21838500 | 22981800 |
| 10851700 | 16066400 | 17434500 | 18527800 | 19443800 | 20806100 | 21840500 | 22984400 |
| 10862500 | 16076200 | 17440300 | 18535800 | 19486900 | 20808400 | 21850900 | 23000600 |
| 10868300 | 16087900 | 17443100 | 18550100 | 19495300 | 20814400 | 21851100 | 23009700 |
| 10889500 | 16099900 | 17456000 | 18570400 | 19495400 | 20825700 | 21851800 | 23012900 |
| 10898800 | 16104500 | 17461800 | 18587100 | 19496300 | 20834300 | 21852800 | 23032500 |
| 10990600 | 16105700 | 17464000 | 18603400 | 19501800 | 20867700 | 21852900 | 23058900 |
| 11021600 | 16120400 | 17464100 | 18611400 | 19506400 | 20869900 | 21856600 | 23061100 |
| 11025700 | 16127400 | 17466600 | 18618800 | 19508200 | 20876400 | 21883000 | 23061600 |
| 11034800 | 16130900 | 17466800 | 18619900 | 19514500 | 20876600 | 21888700 | 23094800 |
| 11048000 | 16141200 | 17467500 | 18622300 | 19523800 | 20889300 | 21891500 | 23095300 |
| 11096200 | 16151900 | 17481900 | 18634200 | 19526200 | 20893200 | 21892000 | 23101600 |
| 11100600 | 16159500 | 17505400 | 18637600 | 19526700 | 20898100 | 21893600 | 23126800 |
| 11106600 | 16163900 | 17506600 | 18643800 | 19530700 | 20900800 | 21900500 | 23129400 |
| 11127600 | 16176600 | 17517700 | 18668800 | 19531400 | 20903700 | 21928500 | 23185500 |
| 11153500 | 16182300 | 17519700 | 18676300 | 19552600 | 20912300 | 21935000 | 23191700 |
| 11161300 | 16218500 | 17528400 | 18677300 | 19562100 | 20920500 | 21938500 | 23196100 |
| 11180200 | 16229300 | 17532700 | 18678900 | 19569200 | 20930800 | 21940100 | 23235500 |
| 14344600 | 16259600 | 17561300 | 18685400 | 19569800 | 20941500 | 21950400 | 23236400 |
| 14361600 | 16260700 | 17561800 | 18699800 | 19591300 | 20944400 | 21965200 | 23240500 |
| 14372500 | 16288000 | 17573800 | 18707300 | 19596200 | 20956500 | 21978100 | 23275900 |
| 14383200 | 16291200 | 17582800 | 18707600 | 19603500 | 20967800 | 21988500 | 23276000 |
| 14390500 | 16307500 | 17584600 | 18715900 | 19613900 | 21013800 | 22001400 | 23290600 |
| 14395600 | 16396400 | 17586100 | 18716200 | 19615600 | 21024900 | 22018800 | 23296900 |
| 14411800 | 16443400 | 17595800 | 18720000 | 19616500 | 21050300 | 22031600 | 23303600 |
| 14431400 | 16452000 | 17619000 | 18740600 | 19619500 | 21074500 | 22043300 | 23326200 |
| 14699500 | 16458600 | 17620100 | 18741900 | 19635700 | 21081100 | 22060800 | 23328300 |
| 14805600 | 16461400 | 17621100 | 18748500 | 19643700 | 21091600 | 22062000 | 23328700 |
| 14828200 | 16518900 | 17627600 | 18778900 | 19649100 | 21101600 | 22080100 | 23338700 |
| 14897900 | 16520800 | 17633400 | 18783100 | 19649900 | 21104100 | 22086700 | 23341100 |
| 14900100 | 16528100 | 17635100 | 18788000 | 19657900 | 21104300 | 22105900 | 23345000 |
| 14944200 | 16553000 | 17637900 | 18797600 | 19688500 | 21108700 | 22106200 | 23354800 |
| 14950900 | 16556500 | 17643000 | 18798400 | 19708400 | 21110400 | 22115900 | 23356100 |
| 15036300 | 16558500 | 17663200 | 18800100 | 19719700 | 21136800 | 22133500 | 23360300 |
| 15054200 | 16565200 | 17666500 | 18819100 | 19727600 | 21140800 | 22134300 | 23365200 |
| 15078000 | 16569700 | 17670500 | 18821500 | 19731400 | 21143400 | 22134400 | 23372200 |
| 15083000 | 16582300 | 17683800 | 18831800 | 19738100 | 21149400 | 22138200 | 23382900 |
| 15087900 | 16604700 | 17698700 | 18834000 | 19743000 | 21151800 | 22144800 | 23389900 |
| 15141900 | 16606800 | 17703300 | 18836800 | 19756000 | 21158900 | 22145200 | 23401700 |
| 15194400 | 16614900 | 17707500 | 18839500 | 19757000 | 21160100 | 22159500 | 23403000 |
| 15255400 | 16628500 | 17709400 | 18848600 | 19759100 | 21167300 | 22161000 | 23404600 |
| 15323900 | 16629000 | 17710800 | 18857500 | 19763800 | 21172800 | 22171200 | 23405000 |
| 15356300 | 16633000 | 17752400 | 18880600 | 19786900 | 21174500 | 22173800 | 23405200 |
| 15372700 | 16643000 | 17758800 | 18900200 | 19794500 | 21183100 | 22174700 | 23407500 |
| 15398100 | 16644600 | 17759500 | 18907500 | 19795600 | 21186600 | 22218100 | 23426000 |
| 15412500 | 16654100 | 17784400 | 18909300 | 19824300 | 21189400 | 22224000 | 23456500 |
| 15434900 | 16685000 | 17788300 | 18910100 | 19824700 | 21189800 | 22234100 | 23467900 |
| 15435600 | 16686400 | 17813400 | 18914600 | 19825200 | 21192600 | 22243700 | 23490200 |
| 15445400 | 16694900 | 17827300 | 18918900 | 19831700 | 21223200 | 22255400 | 23492700 |
| 15451600 | 16707300 | 17828200 | 18921800 | 19837300 | 21224300 | 22264200 | 23502200 |
| 15528500 | 16732500 | 17832300 | 18942200 | 19849300 | 21226200 | 22265000 | 23507500 |
| 15546200 | 16744000 | 17838700 | 18943400 | 19858300 | 21233900 | 22270700 | 23510800 |
| 15576100 | 16756800 | 17856200 | 18951300 | 19859200 | 21238500 | 22278500 | 23534900 |
| 15589600 | 16777100 | 17864500 | 18955000 | 19865900 | 21239300 | 22280600 | 23576400 |
| 15607200 | 16786100 | 17864900 | 18956800 | 19867200 | 21273800 | 22281500 | 23582100 |
| 15608600 | 16798600 | 17875800 | 18998400 | 19890600 | 21282600 | 22281800 | 23611500 |
| 15609000 | 16859000 | 17877800 | 18999200 | 19891100 | 21283300 | 22294100 | 23657500 |
| 15615500 | 16860400 | 17885300 | 19000900 | 19903500 | 21301000 | 22312200 | 23659400 |
| 15620000 | 16863700 | 17896400 | 19010800 | 19911700 | 21304400 | 22313200 | 23667100 |
| 15629000 | 16872300 | 17898500 | 19023900 | 19912700 | 21310100 | 22319300 | 23672700 |
| 15629700 | 16875900 | 17914500 | 19026000 | 19940300 | 21316200 | 22323700 | 23696400 |
| 15630300 | 16885500 | 17916200 | 19030300 | 19952500 | 21326500 | 22325600 | 23718600 |
| 15633600 | 16892300 | 17935900 | 19041100 | 19979900 | 21333300 | 22331400 | 23779300 |
| 15639900 | 16894100 | 17939600 | 19044700 | 19985200 | 21354100 | 22333100 | 23793400 |
| 15641300 | 16904300 | 17961100 | 19047700 | 20009000 | 21354200 | 22337300 | 23803600 |
| 15646800 | 16922200 | 17976600 | 19049500 | 20015100 | 21365300 | 22337800 | 23808300 |
| 15650300 | 16932600 | 17982200 | 19053900 | 20024300 | 21366400 | 22347600 | 23826100 |
| 15665500 | 16934400 | 17985200 | 19054300 | 20120500 | 21381000 | 22347900 | 23831100 |
| 15670200 | 16936900 | 17997200 | 19063100 | 20120600 | 21388800 | 22351600 | 23833100 |
| 15673600 | 16942700 | 18009700 | 19082400 | 20128100 | 21400400 | 22356700 | 23833700 |
| 15676400 | 16943100 | 18023100 | 19095100 | 20131000 | 21404000 | 22371200 | 23833900 |
| 15686900 | 16963300 | 18032400 | 19098100 | 20131500 | 21415000 | 22378400 | 23835900 |
| 15687000 | 16964400 | 18038700 | 19100300 | 20149600 | 21416100 | 22381400 | 23840200 |
| 15703200 | 16967500 | 18040100 | 19108000 | 20180700 | 21416900 | 22383100 | 23853500 |
| 15709200 | 16969300 | 18049300 | 19110700 | 20200400 | 21433900 | 22385800 | 23859400 |
| 15709900 | 16979600 | 18054800 | 19114200 | 20208800 | 21436900 | 22392900 | 23871200 |
| 15713500 | 16996400 | 18077000 | 19117100 | 20227000 | 21454900 | 22402900 | 23876400 |
| 15715700 | 16997200 | 18086100 | 19117200 | 20239300 | 21460100 | 22423000 | 23877000 |
| 15717600 | 17008700 | 18097800 | 19117800 | 20239400 | 21467700 | 22456400 | 23880000 |
| 15724200 | 17012700 | 18102600 | 19117900 | 20260500 | 21475300 | 22459000 | 23890200 |
| 15741400 | 17017800 | 18109800 | 19119800 | 20268300 | 21476300 | 22462200 | 23890500 |
| 15757600 | 17025900 | 18118400 | 19132800 | 20286400 | 21482800 | 22477300 | 23891200 |
| 15760000 | 17030000 | 18127000 | 19147700 | 20289300 | 21490300 | 22478300 | 23901600 |
| 15770300 | 17062200 | 18145400 | 19203900 | 20295300 | 21493900 | 22482400 | 23917700 |
| 15782400 | 17063900 | 18154600 | 19205400 | 20320600 | 21496200 | 22495500 | 23918900 |
| 15791300 | 17064700 | 18157800 | 19205800 | 20322700 | 21496700 | 22498300 | 23923900 |
| 15806500 | 17074500 | 18163100 | 19205900 | 20326300 | 21499100 | 22519200 | 23931300 |
| 15807300 | 17079000 | 18168000 | 19206700 | 20337600 | 21501000 | 22534100 | 23934300 |
| 15810000 | 17086600 | 18169800 | 19218500 | 20349500 | 21501100 | 22547500 | 23941700 |
| 15811800 | 17090900 | 18172800 | 19219800 | 20353400 | 21509600 | 22562200 | 23950900 |
| 15820300 | 17094100 | 18179000 | 19221900 | 20356400 | 21516800 | 22582000 | 23951200 |
| 15833000 | 17101300 | 18187900 | 19222000 | 20362800 | 21522400 | 22606900 | 23952600 |
| 15834600 | 17103200 | 18196800 | 19223900 | 20364900 | 21533000 | 22607100 | 23959700 |
| 15838300 | 17111700 | 18200900 | 19248800 | 20394300 | 21548500 | 22615800 | 23972500 |
| 15839100 | 17116600 | 18217200 | 19251000 | 20395900 | 21567700 | 22631300 | 23981400 |
| 15845600 | 17133800 | 18221600 | 19252500 | 20429600 | 21594700 | 22646300 | 23983100 |
| 15853600 | 17154600 | 18223800 | 19256700 | 20436900 | 21616500 | 22687500 | 23992300 |
| 15866100 | 17160400 | 18233400 | 19274300 | 20453600 | 21636700 | 22690100 | 23999500 |
| 15876600 | 17207100 | 18251500 | 19288100 | 20462000 | 21636900 | 22697000 | 24018400 |
| 15882100 | 17218700 | 18252100 | 19288200 | 20476500 | 21637100 | 22701400 | 24018800 |
| 15899800 | 17276800 | 18259400 | 19296000 | 20500600 | 21643600 | 22744100 | 24025300 |
| 15909400 | 17278400 | 18267400 | 19302000 | 20506900 | 21670800 | 22745700 | 24057800 |
| 15928100 | 17279200 | 18274100 | 19305800 | 20519200 | 21672100 | 22754900 | 24061000 |
| 15936100 | 17285600 | 18301200 | 19317400 | 20536400 | 21678300 | 22762300 | 24074500 |
| 15941400 | 17292700 | 18310100 | 19328300 | 20548000 | 21678700 | 22769500 | 24084900 |
| 15947900 | 17296700 | 18315000 | 19334800 | 20566900 | 21679300 | 22773000 | 24089300 |
| 15955700 | 17300600 | 18317500 | 19335700 | 20581300 | 21691700 | 22790800 | 24105100 |
| 15970000 | 17303600 | 18322500 | 19346600 | 20591500 | 21714200 | 22809100 | 24113400 |
| 15972100 | 17315700 | 18325500 | 19354400 | 20608700 | 21719300 | 22809300 | 24114000 |
| 15982800 | 17320300 | 18334900 | 19375100 | 20614800 | 21739500 | 22818500 | 24139100 |

The selected T21-specific u-DLRs

| | | | | | | | |
|---|---|---|---|---|---|---|---|
| 15078000 | 20843100 | 24937300 | 31821900 | 34672900 | 42770000 | 47588600 | 38660800 |
| 15413700 | 21451400 | 25752700 | 32258800 | 34690700 | 43291800 | 9875200 | 41842500 |
| 15486300 | 21739100 | 25887700 | 32294400 | 34872200 | 43644200 | 18679500 | 45355100 |
| 15490100 | 21771600 | 26081000 | 32526200 | 35234300 | 43933400 | 22295500 | 45734900 |
| 15680600 | 22449400 | 28463100 | 32748600 | 36191800 | 44303300 | 22450800 | 45770300 |
| 16547900 | 22459500 | 29713600 | 32900300 | 36193500 | 44303600 | 26152100 | 45946100 |
| 17461600 | 22530700 | 29732000 | 33572800 | 37070300 | 45151100 | 31408200 | 46316400 |
| 18123400 | 22715800 | 29879100 | 33831600 | 38032500 | 45597700 | 32639100 | 48079600 |
| 18499700 | 22908900 | 31306700 | 33875700 | 39652400 | 45708400 | 33533900 | |
| 19286900 | 22921700 | 31357700 | 33919200 | 40405900 | 46009400 | 33591700 | |
| 20037100 | 23004300 | 31489300 | 34092400 | 41285400 | 46780500 | 33840400 | |
| 20042500 | 23380000 | 31568000 | 34460800 | 42378900 | 47329500 | 36220800 | |

TABLE 5
[The coordinate is shown for the first base pair of 100 bp hm-DLRs in chromosome 21]
Pregnancy-specific hm-DLRs

| | | | | | | | |
|---|---|---|---|---|---|---|---|
| 15078000 | 30594600 | 35231100 | 38321300 | 42695100 | 44301100 | 45492400 | 46747600 |
| 15442500 | 30642600 | 35246100 | 38335200 | 42738300 | 44326600 | 45494400 | 46748200 |
| 15496700 | 30658000 | 35272400 | 38441400 | 42746900 | 44329100 | 45498400 | 46748800 |
| 15970900 | 30669700 | 35293400 | 38443100 | 42760300 | 44334600 | 45542100 | 46769800 |
| 16119600 | 30675000 | 35344900 | 38454300 | 42824800 | 44347000 | 45560600 | 46776900 |
| 16193000 | 30708600 | 35349600 | 38541100 | 42851600 | 44350500 | 45568800 | 46777600 |
| 16213800 | 30719600 | 35444700 | 38566700 | 42860000 | 44354200 | 45571400 | 46780700 |
| 16214600 | 30755900 | 35500600 | 38567300 | 42860500 | 44361200 | 45572100 | 46784600 |
| 16240400 | 31030700 | 35516100 | 38579100 | 42874000 | 44366300 | 45614500 | 46790700 |
| 16311800 | 31223600 | 35560500 | 38634900 | 43030200 | 44381700 | 45621700 | 46799200 |
| 16326500 | 32471400 | 35587700 | 38636200 | 43050800 | 44383700 | 45630000 | 46869900 |
| 16389000 | 32510000 | 35616700 | 38662600 | 43092100 | 44387300 | 45632500 | 46870200 |
| 16395200 | 32575500 | 35712600 | 38676800 | 43115200 | 44387900 | 45637600 | 46886600 |
| 16396200 | 32581400 | 35718200 | 38732000 | 43135400 | 44442400 | 45658000 | 46902700 |
| 16407900 | 32678200 | 35755000 | 38750600 | 43154800 | 44448300 | 45659300 | 46911600 |
| 16488800 | 32711300 | 35761500 | 38766900 | 43171100 | 44461800 | 45663300 | 46914600 |
| 16511900 | 32725600 | 35879600 | 38767300 | 43172900 | 44467700 | 45675100 | 46924300 |
| 16572400 | 32831900 | 35884200 | 38822100 | 43175500 | 44475900 | 45704700 | 46925400 |
| 16572800 | 32840800 | 35886800 | 38832800 | 43175800 | 44491400 | 45705900 | 46931500 |
| 16582800 | 32898600 | 35893700 | 38888500 | 43179100 | 44508400 | 45724700 | 46932100 |
| 16591800 | 32915200 | 35894400 | 38890600 | 43228700 | 44573600 | 45743000 | 46932200 |
| 16643600 | 32915700 | 35922100 | 38920900 | 43228800 | 44591600 | 45747000 | 46932700 |
| 16682300 | 32934700 | 35940800 | 38942100 | 43239800 | 44594100 | 45751700 | 46934200 |
| 16684400 | 32986500 | 35963200 | 38964000 | 43241000 | 44595900 | 45773900 | 46945700 |
| 16706000 | 32999300 | 36072400 | 39104900 | 43242000 | 44614600 | 45796000 | 46950400 |
| 16763200 | 33005100 | 36076400 | 39343900 | 43245800 | 44626800 | 45825100 | 46959000 |
| 16828600 | 33012700 | 36079700 | 39461200 | 43256600 | 44704800 | 45826500 | 46959800 |
| 16884200 | 33019200 | 36081500 | 39490900 | 43293000 | 44732600 | 45843300 | 46971900 |
| 16888000 | 33026800 | 36108600 | 39594800 | 43314600 | 44762100 | 45880200 | 46973300 |
| 17036800 | 33057900 | 36157900 | 39598900 | 43319100 | 44782500 | 45883300 | 46977800 |
| 17086600 | 33085600 | 36164200 | 39632900 | 43319500 | 44784900 | 45898800 | 46980800 |
| 17099100 | 33671200 | 36165000 | 39706900 | 43344400 | 44802600 | 45928000 | 47051900 |
| 17099800 | 33723100 | 36198900 | 39755900 | 43351200 | 44814600 | 45956000 | 47124500 |
| 17117000 | 33725000 | 36202900 | 39761300 | 43376500 | 44817100 | 46034900 | 47188800 |
| 17193500 | 33763500 | 36208100 | 39851400 | 43384100 | 44837100 | 46055600 | 47239300 |
| 17550000 | 33792500 | 36242900 | 39948700 | 43394500 | 44870700 | 46063700 | 47251700 |
| 17561300 | 33805800 | 36243000 | 39970500 | 43412800 | 44871500 | 46068700 | 47288700 |
| 17578000 | 33823700 | 36244900 | 39984000 | 43443300 | 44872300 | 46142700 | 47290300 |
| 17592700 | 33857800 | 36246600 | 40119400 | 43446200 | 44876300 | 46154200 | 47333300 |
| 17666600 | 33881100 | 36288500 | 40123900 | 43456200 | 44883100 | 46182800 | 47403100 |
| 17734100 | 33901800 | 36329200 | 40134200 | 43499900 | 44883700 | 46214100 | 47418400 |
| 18846300 | 33948600 | 36331900 | 40166900 | 43506100 | 44900600 | 46235600 | 47422300 |
| 18857600 | 33957500 | 36345600 | 40176200 | 43567000 | 44916900 | 46253400 | 47423800 |
| 18883100 | 33966100 | 36389700 | 40244600 | 43571300 | 44924000 | 46270000 | 47457000 |
| 19052700 | 34062900 | 36444900 | 40277900 | 43577500 | 44928000 | 46271300 | 47515500 |
| 19066500 | 34069100 | 36595800 | 40285300 | 43603000 | 44928800 | 46272800 | 47520800 |
| 19069400 | 34075400 | 36656600 | 40293700 | 43621300 | 44935600 | 46284800 | 47530400 |
| 19071700 | 34185900 | 36693800 | 40293800 | 43679800 | 45039800 | 46286600 | 47538400 |
| 19106400 | 34338600 | 36829500 | 40310600 | 43681500 | 45040100 | 46307200 | 47541800 |
| 19118800 | 34402600 | 36840000 | 40349000 | 43786800 | 45064600 | 46308100 | 47542400 |
| 19150100 | 34405900 | 36944400 | 40352500 | 43790900 | 45067700 | 46319500 | 47545100 |
| 19173600 | 34409600 | 37015500 | 40356600 | 43801700 | 45092200 | 46320800 | 47552700 |
| 19176300 | 34447800 | 37033700 | 40357800 | 43813600 | 45105500 | 46326500 | 47556200 |
| 19228600 | 34477900 | 37038600 | 40358000 | 43817900 | 45109700 | 46328000 | 47574100 |
| 21311300 | 34517400 | 37169900 | 40372800 | 43844600 | 45116700 | 46349100 | 47577700 |
| 21626600 | 34556200 | 37277600 | 40394400 | 43846300 | 45129900 | 46371800 | 47608400 |
| 22421400 | 34618500 | 37334100 | 40395100 | 43846800 | 45131700 | 46379600 | 47617600 |
| 23735900 | 34623200 | 37436800 | 40453300 | 43869600 | 45147000 | 46396500 | 47624100 |
| 25184700 | 34625700 | 37456000 | 40466200 | 43872000 | 45153200 | 46398100 | 47630200 |
| 25711300 | 34638000 | 37459800 | 40466900 | 43893300 | 45182000 | 46401200 | 47631900 |
| 27006500 | 34643100 | 37537100 | 40479000 | 43896600 | 45190800 | 46403700 | 47632900 |
| 27157900 | 34717700 | 37542300 | 40479900 | 43898300 | 45191200 | 46407900 | 47676300 |
| 27190500 | 34722900 | 37554900 | 40542000 | 43915300 | 45228200 | 46412700 | 47686900 |
| 27287800 | 34728000 | 37559100 | 40568900 | 43943900 | 45229000 | 46442000 | 47700500 |
| 27332200 | 34753100 | 37609600 | 40637700 | 43977800 | 45232900 | 46449800 | 47715600 |
| 27397400 | 34754700 | 37627900 | 40730800 | 43988800 | 45234200 | 46451400 | 47764000 |
| 27424500 | 34756700 | 37639400 | 40741800 | 44003400 | 45242100 | 46455200 | 47766000 |
| 27434400 | 34774100 | 37646900 | 40763200 | 44004600 | 45244300 | 46455400 | 47780500 |
| 27445300 | 34790400 | 37658400 | 40773600 | 44006200 | 45246200 | 46461600 | 47786500 |
| 27449100 | 34790500 | 37674700 | 40815500 | 44033500 | 45253700 | 46473600 | 47793600 |
| 27452300 | 34811500 | 37750300 | 40841700 | 44037100 | 45271700 | 46480500 | 47805500 |
| 27489500 | 34814600 | 37758700 | 40881500 | 44053600 | 45286500 | 46491900 | 47861300 |
| 27559600 | 34848800 | 37772200 | 41010600 | 44064900 | 45298300 | 46560000 | 47939900 |
| 27895000 | 34911100 | 37791900 | 41086000 | 44075700 | 45298700 | 46566300 | 47946100 |
| 27938500 | 34923100 | 37795000 | 41130500 | 44115900 | 45299700 | 46568900 | 47976300 |
| 28256200 | 35023200 | 37819200 | 41132500 | 44117300 | 45325300 | 46640200 | 47980600 |
| 28307900 | 35047800 | 37978700 | 41919500 | 44144600 | 45338100 | 46643200 | 47983300 |
| 28515800 | 35058800 | 38028500 | 42036400 | 44152000 | 45343000 | 46677400 | 47985500 |
| 29484500 | 35065700 | 38060900 | 42419200 | 44173600 | 45364400 | 46677800 | 47985700 |
| 30006300 | 35142600 | 38100300 | 42442800 | 44182200 | 45373900 | 46683700 | 48024400 |
| 30241900 | 35169600 | 38140400 | 42543300 | 44255200 | 45396700 | 46685300 | 48041700 |
| 30436300 | 35201900 | 38153300 | 42546300 | 44281900 | 45431100 | 46699900 | 48048700 |
| 30494600 | 35203700 | 38192600 | 42551900 | 44282200 | 45446700 | 46700100 | 48054400 |
| 30535200 | 35217300 | 38215600 | 42595400 | 44282300 | 45448900 | 46715700 | 48070800 |
| 30536400 | 35227600 | 38282500 | 42625000 | 44300500 | 45470500 | 46728100 | |

Tissue-specific hm-DLRs. Only 1000 selected DLRs are shown

| | | | | | | | |
|---|---|---|---|---|---|---|---|
| 10421900 | 16261100 | 16954300 | 17764700 | 18920700 | 23068700 | 27303600 | 27558300 |
| 10589700 | 16261600 | 16956100 | 17764900 | 18921000 | 23227200 | 27303900 | 27558700 |
| 10596500 | 16262900 | 16956500 | 17766800 | 18923400 | 23236000 | 27304800 | 27559900 |
| 10596600 | 16274900 | 16962500 | 17767800 | 18926900 | 23291400 | 27306000 | 27562900 |
| 10598400 | 16283000 | 16975500 | 17773300 | 18931000 | 23456600 | 27307500 | 27566800 |
| 10598900 | 16284300 | 16976200 | 17785700 | 18943000 | 23492500 | 27307800 | 27569500 |
| 10715400 | 16289300 | 16976400 | 17796400 | 18952700 | 23492900 | 27307900 | 27570000 |
| 10736200 | 16291100 | 16989000 | 17799200 | 18956300 | 23510900 | 27308200 | 27577000 |
| 10843800 | 16291200 | 16989500 | 17813700 | 18956800 | 23518700 | 27316600 | 27580900 |
| 10913500 | 16299300 | 16990900 | 17886800 | 18968000 | 23525700 | 27324600 | 27581400 |
| 10924600 | 16299800 | 16991500 | 17898600 | 18970500 | 23528800 | 27326100 | 27585800 |
| 10955200 | 16311900 | 16992000 | 17905500 | 18971800 | 23552200 | 27327900 | 27593300 |
| 10987700 | 16325200 | 16997600 | 17905800 | 18973800 | 23560200 | 27330800 | 27600300 |
| 10992800 | 16326500 | 17001900 | 17906900 | 18975600 | 23562800 | 27335500 | 27602200 |
| 11012100 | 16329200 | 17009300 | 17909300 | 18977100 | 23573000 | 27335700 | 27604900 |
| 11028500 | 16329400 | 17017900 | 17922600 | 18977300 | 23573400 | 27337100 | 27605000 |
| 11094600 | 16332100 | 17034600 | 17924100 | 18982100 | 23616700 | 27338200 | 27605300 |
| 11098900 | 16334600 | 17036800 | 17928200 | 18987300 | 23629800 | 27340000 | 27607200 |
| 11112100 | 16347100 | 17040700 | 17928300 | 19005000 | 23656900 | 27340500 | 27609200 |
| 11122000 | 16357400 | 17041300 | 17928400 | 19010300 | 23659900 | 27341200 | 27609700 |
| 11130500 | 16366000 | 17041400 | 17928900 | 19020100 | 23667300 | 27342900 | 27610200 |
| 11131500 | 16373300 | 17041700 | 17931600 | 19023400 | 23682600 | 27343100 | 27617500 |
| 11132500 | 16373500 | 17045000 | 17934400 | 19028400 | 23701700 | 27343200 | 27620800 |
| 11139800 | 16373700 | 17045100 | 17936200 | 19031000 | 23724200 | 27351000 | 27621100 |
| 11144000 | 16374700 | 17046400 | 17936400 | 19033300 | 23732400 | 27352700 | 27625200 |
| 11144200 | 16380800 | 17049500 | 17941100 | 19033700 | 23732500 | 27354700 | 27626300 |
| 11145700 | 16382700 | 17050200 | 17943400 | 19033900 | 23732700 | 27360300 | 27631000 |
| 11170400 | 16383100 | 17065200 | 17944100 | 19034800 | 23735900 | 27360700 | 27637700 |
| 14384400 | 16388400 | 17080100 | 17945100 | 19035000 | 23768500 | 27362800 | 27656300 |
| 14804300 | 16391900 | 17080600 | 17945800 | 19041900 | 23811300 | 27363900 | 27656400 |
| 14816400 | 16396200 | 17084900 | 17945900 | 19042100 | 23833900 | 27369600 | 27658700 |
| 15056300 | 16396800 | 17085500 | 17947600 | 19045100 | 23918100 | 27371100 | 27659000 |
| 15067900 | 16399900 | 17089800 | 17956700 | 19045800 | 23947700 | 27372700 | 27691600 |
| 15068200 | 16400700 | 17094100 | 17958000 | 19046600 | 23950600 | 27373700 | 27693400 |
| 15077900 | 16401600 | 17099100 | 17958400 | 19048800 | 24744100 | 27375500 | 27697600 |
| 15166800 | 16401700 | 17099300 | 17961700 | 19052000 | 24825100 | 27375800 | 27718300 |
| 15227100 | 16407000 | 17116900 | 17965900 | 19052600 | 24974800 | 27376700 | 27744600 |
| 15228900 | 16407900 | 17117000 | 17971500 | 19063900 | 25255800 | 27377900 | 27760700 |
| 15261500 | 16423400 | 17121500 | 17978700 | 19070600 | 25258400 | 27381200 | 27763400 |
| 15261700 | 16423900 | 17123000 | 17979900 | 19071100 | 25301000 | 27382900 | 27763500 |
| 15262000 | 16425600 | 17127100 | 18023400 | 19077100 | 25304000 | 27383300 | 27765300 |
| 15297900 | 16426600 | 17142200 | 18029900 | 19098900 | 25370100 | 27384900 | 27765800 |
| 15300600 | 16428100 | 17145100 | 18040100 | 19100300 | 25580300 | 27387100 | 27766600 |
| 15309200 | 16429800 | 17147400 | 18042800 | 19101100 | 25871200 | 27388300 | 27766800 |
| 15357200 | 16433200 | 17153200 | 18049200 | 19102500 | 26100000 | 27389600 | 27769700 |
| 15375900 | 16434800 | 17154000 | 18078000 | 19104300 | 26219400 | 27397000 | 27770900 |
| 15380100 | 16434900 | 17156500 | 18078200 | 19104400 | 26335300 | 27397400 | 27773000 |
| 15381100 | 16435500 | 17157700 | 18085600 | 19108200 | 26656500 | 27399800 | 27775500 |
| 15383400 | 16435600 | 17166300 | 18141500 | 19108800 | 26833400 | 27407900 | 27776200 |
| 15383800 | 16438600 | 17172100 | 18144200 | 19116400 | 26929000 | 27410800 | 27776400 |
| 15384000 | 16444100 | 17174000 | 18147500 | 19116800 | 26930500 | 27411200 | 27776700 |
| 15384700 | 16444300 | 17176800 | 18215200 | 19117200 | 26932600 | 27411900 | 27777100 |
| 15386000 | 16451600 | 17178500 | 18443200 | 19117800 | 26934200 | 27414400 | 27778500 |
| 15386300 | 16467200 | 17180200 | 18699900 | 19119800 | 26935800 | 27417100 | 27779300 |
| 15404000 | 16469800 | 17182400 | 18762800 | 19128600 | 26940700 | 27417600 | 27780400 |
| 15407300 | 16478900 | 17182500 | 18763400 | 19131400 | 26942300 | 27424500 | 27783600 |
| 15412600 | 16479500 | 17187400 | 18766800 | 19136100 | 26945900 | 27428100 | 27783800 |
| 15431700 | 16491000 | 17188500 | 18772200 | 19150100 | 26948800 | 27428400 | 27784100 |
| 15434600 | 16494700 | 17193500 | 18782400 | 19151900 | 26961400 | 27430700 | 27796700 |
| 15435600 | 16504800 | 17193700 | 18782600 | 19162200 | 26971700 | 27431100 | 27799400 |
| 15436100 | 16505800 | 17197300 | 18788400 | 19166300 | 26973100 | 27431200 | 27812100 |
| 15436700 | 16506400 | 17206900 | 18793000 | 19167200 | 26978700 | 27434300 | 27817200 |
| 15436900 | 16506500 | 17207100 | 18793300 | 19167300 | 26980800 | 27440500 | 27818000 |
| 15442600 | 16507400 | 17210000 | 18807500 | 19173600 | 26986400 | 27440700 | 27818500 |
| 15442700 | 16510800 | 17211500 | 18808900 | 19174700 | 26986600 | 27443300 | 27822300 |
| 15443000 | 16511000 | 17212800 | 18809700 | 19175600 | 26997800 | 27443400 | 27823900 |
| 15443100 | 16521700 | 17213100 | 18809900 | 19177800 | 26998000 | 27445200 | 27825700 |
| 15444800 | 16522600 | 17218600 | 18810300 | 19188900 | 26998200 | 27446900 | 27827800 |
| 15447000 | 16536900 | 17221000 | 18812500 | 19196300 | 27003800 | 27448400 | 27831100 |
| 15448400 | 16547800 | 17222100 | 18813900 | 19196900 | 27020000 | 27449100 | 27835300 |
| 15451300 | 16551900 | 17226200 | 18816600 | 19202000 | 27038000 | 27449300 | 27838000 |
| 15452900 | 16560600 | 17232500 | 18817500 | 19213400 | 27050200 | 27450800 | 27840200 |
| 15453000 | 16569600 | 17236600 | 18817600 | 19228600 | 27054700 | 27452300 | 27840900 |
| 15455900 | 16572400 | 17247200 | 18818700 | 19278100 | 27055500 | 27459000 | 27843400 |
| 15456600 | 16574500 | 17247400 | 18820500 | 19280100 | 27072600 | 27463900 | 27846500 |
| 15457200 | 16577000 | 17261000 | 18821800 | 19294600 | 27072900 | 27465900 | 27855900 |
| 15458000 | 16581900 | 17268600 | 18822500 | 19311000 | 27090600 | 27467700 | 27857900 |
| 15464500 | 16585300 | 17277600 | 18822900 | 19318500 | 27094500 | 27468400 | 27867400 |
| 15464700 | 16591800 | 17279700 | 18823100 | 19345900 | 27098000 | 27468500 | 27868200 |
| 15465300 | 16592400 | 17280700 | 18824500 | 19514500 | 27098200 | 27469200 | 27874100 |
| 15468700 | 16592500 | 17300600 | 18826000 | 19764200 | 27098400 | 27470200 | 27874900 |
| 15471100 | 16600400 | 17305200 | 18827900 | 20037300 | 27102000 | 27471200 | 27875500 |
| 15473800 | 16611500 | 17305400 | 18828300 | 20173200 | 27109900 | 27476700 | 27877200 |
| 15474300 | 16615800 | 17333200 | 18829500 | 20216000 | 27122500 | 27477300 | 27887800 |
| 15474400 | 16615900 | 17341500 | 18829800 | 20250500 | 27127200 | 27479800 | 27889100 |
| 15477900 | 16620100 | 17350300 | 18831300 | 20270100 | 27127300 | 27481500 | 27903600 |
| 15491600 | 16625100 | 17352300 | 18834100 | 20508400 | 27135600 | 27485300 | 27923600 |
| 15491800 | 16627700 | 17353900 | 18834500 | 20649200 | 27140300 | 27489700 | 27942700 |
| 15552200 | 16633100 | 17354100 | 18834600 | 20966100 | 27157500 | 27490800 | 27945000 |
| 15647300 | 16647900 | 17356000 | 18835600 | 21390900 | 27161300 | 27491000 | 27958500 |
| 15650300 | 16663300 | 17357600 | 18842600 | 21540500 | 27173500 | 27493200 | 27960200 |
| 15705500 | 16664500 | 17362400 | 18843000 | 21546100 | 27184800 | 27495100 | 27960300 |
| 15731300 | 16670800 | 17363500 | 18848200 | 21594000 | 27185000 | 27497800 | 27963700 |
| 15734600 | 16672300 | 17371300 | 18849100 | 22347600 | 27185100 | 27498300 | 28021600 |
| 15743100 | 16673600 | 17376500 | 18853800 | 22367200 | 27190500 | 27500200 | 28026000 |
| 15748000 | 16677500 | 17377500 | 18854600 | 22369700 | 27192700 | 27502400 | 28027800 |
| 15748600 | 16688000 | 17422500 | 18858800 | 22370000 | 27194600 | 27503000 | 28031600 |
| 15758400 | 16710100 | 17433700 | 18861900 | 22370200 | 27200700 | 27503200 | 28041800 |
| 15765100 | 16717600 | 17442900 | 18862400 | 22381400 | 27207100 | 27504200 | 28047900 |
| 15807300 | 16729800 | 17443400 | 18864000 | 22386400 | 27207500 | 27505400 | 28049900 |
| 15811600 | 16732800 | 17457400 | 18865600 | 22396900 | 27208700 | 27508300 | 28051800 |
| 15851300 | 16769900 | 17485500 | 18866400 | 22397500 | 27213300 | 27509100 | 28056300 |
| 15854600 | 16799800 | 17485700 | 18867100 | 22399400 | 27214200 | 27509200 | 28074300 |
| 15869800 | 16815100 | 17489400 | 18867400 | 22413800 | 27217000 | 27510800 | 28075700 |
| 15983000 | 16816700 | 17496000 | 18868000 | 22421500 | 27218200 | 27511400 | 28080900 |
| 16007100 | 16818200 | 17505000 | 18868600 | 22429800 | 27232100 | 27511500 | 28081500 |
| 16016200 | 16828600 | 17541600 | 18870800 | 22440800 | 27243600 | 27518500 | 28081900 |
| 16105700 | 16842900 | 17542100 | 18872900 | 22452600 | 27247400 | 27518800 | 28093200 |
| 16197300 | 16854200 | 17544000 | 18873100 | 22461200 | 27252100 | 27519700 | 28094900 |
| 16202200 | 16855700 | 17552100 | 18876400 | 22493100 | 27256300 | 27522300 | 28095900 |
| 16213600 | 16859000 | 17552200 | 18878800 | 22514900 | 27258500 | 27522600 | 28100300 |
| 16213800 | 16866200 | 17565400 | 18879100 | 22537800 | 27260300 | 27524100 | 28104200 |
| 16213900 | 16867200 | 17568100 | 18880800 | 22537900 | 27261700 | 27524400 | 28105700 |
| 16214600 | 16872200 | 17568500 | 18883100 | 22555600 | 27275300 | 27527500 | 28105800 |
| 16215100 | 16872400 | 17612000 | 18891400 | 22564300 | 27276400 | 27528200 | 28106000 |
| 16222100 | 16893900 | 17636600 | 18894500 | 22572100 | 27277200 | 27529400 | 28106900 |
| 16240400 | 16894500 | 17651900 | 18894600 | 22573400 | 27278600 | 27529900 | 28107200 |
| 16241200 | 16914400 | 17653200 | 18894900 | 22591200 | 27281000 | 27534800 | 28107900 |
| 16248200 | 16915700 | 17659500 | 18895500 | 22619600 | 27281400 | 27537800 | 28108900 |
| 16248900 | 16916300 | 17675200 | 18901000 | 22620800 | 27281900 | 27537900 | 28109400 |
| 16249300 | 16932100 | 17675400 | 18902800 | 22631200 | 27282500 | 27538800 | 28109800 |
| 16251500 | 16936900 | 17690400 | 18904100 | 22640300 | 27298500 | 27544700 | 28110400 |
| 16253600 | 16940600 | 17728700 | 18908700 | 22651800 | 27298600 | 27551800 | 28112300 |
| 16255000 | 16948100 | 17741300 | 18912400 | 22728200 | 27300300 | 27552500 | 28114800 |
| 16260700 | 16952900 | 17763100 | 18913800 | 22737400 | 27303000 | 27553700 | 28114900 |

The selected T21-specific hm-DLRs

| | | | | | | | |
|---|---|---|---|---|---|---|---|
| 27300500 | 35948700 | 39631800 | 43336700 | 45754400 | 48054800 | 40036100 | 45715400 |
| 27447600 | 36053400 | 39790900 | 43722500 | 46170300 | 15496700 | 40305700 | 45747100 |
| 30341400 | 36175700 | 39841200 | 43763200 | 46261400 | 15841200 | 40411000 | 45884900 |
| 30692000 | 36185500 | 40204700 | 43896900 | 46387900 | 16481500 | 42682100 | 46235600 |
| 32936900 | 36215200 | 40303500 | 44427300 | 46551600 | 20885400 | 43256500 | 46463000 |
| 32942700 | 36381000 | 40340900 | 44511200 | 46984700 | 34790500 | 43319100 | 46851000 |
| 33019400 | 37847300 | 40704800 | 44615300 | 47183600 | 35616700 | 43418800 | 46932200 |
| 33801400 | 38262700 | 40717600 | 44906600 | 47707300 | 35894400 | 43932600 | 47983300 |
| 34419100 | 38327400 | 40973900 | 44916000 | 47844900 | 35913900 | 44775800 | |
| 35203200 | 38434500 | 42694700 | 45546000 | 47897800 | 35936600 | 45244300 | |
| 35937700 | 39484900 | 43127700 | 45753000 | 47947900 | 35948600 | 45331400 | |

TABLE 6
Selected u-CG-DLRs and hm-CG-DLRs for
fetal T21 and fetal gender determination

| DLR type | | Position |
|---|---|---|
| Detection of fetal T21 aneuploidy | uCG | chr21 29732020 |
| Detection of fetal T21 aneuploidy | uCG | chr21 33462648 |
| Detection of fetal T21 aneuploidy | uCG | chr21 34672959 |
| Detection of fetal T21 aneuploidy | uCG | chr21 36193512 |
| Detection of fetal T21 aneuploidy | uCG | chr21 40801830 |
| Detection of fetal T21 aneuploidy | uCG | chr21 44303692 |
| Detection of fetal T21 aneuploidy | uCG | chr21 44741616 |
| Detection of fetal T21 aneuploidy | uCG | chr21 45798427 |
| Detection of fetal T21 aneuploidy | hmCG | chr21 30341466 |
| Detection of fetal T21 aneuploidy | hmCG | chr21 35898716 |
| Detection of fetal T21 aneuploidy | hmCG | chr21 38327475 |
| Detection of fetal T21 aneuploidy | hmCG | chr21 40074274 |
| Detection of fetal T21 aneuploidy | hmCG | chr21 40135661 |
| Detection of fetal T21 aneuploidy | hmCG | chr21 44084933 |
| Detection of fetal T21 aneuploidy | hmCG | chr21 45546038 |
| Detection of fetal T21 aneuploidy | hmCG | chr21 46964859 |
| Fetal gender determination | uCG | chrX 22425661 |
| Fetal gender determination | uCG | chrX 50774868 |
| Fetal gender determination | uCG | chrX 23776534 |
| Fetal gender determination | uCG | chrX 9624546 |
| Fetal gender determination | uCG | chrX 9389347 |
| Fetal gender determination | uCG | chrX 62584036 |
| Fetal gender determination | uCG | chrX 138802442 |
| Fetal gender determination | uCG | chrY 14774154 |

TABLE 7
[The list of selected DLRs in chromosome 13 and 18. The coordinate
is shown for the first base pair of 100 bp u-DLRs and hm-DLRs]
Pregnancy-specific chr13 u-DLRs

| | | | | | | | |
|---|---|---|---|---|---|---|---|
| 100008100 | 101482600 | 104426200 | 108936500 | 110709500 | 112672700 | 113632800 | 113742100 |
| 100038800 | 101710500 | 104949900 | 109038400 | 110846700 | 112681700 | 113649800 | 113761500 |
| 100066600 | 101742200 | 105608400 | 109386000 | 111057200 | 112690800 | 113653700 | 114185600 |
| 100315300 | 101779900 | 106272200 | 109429500 | 111090000 | 113103000 | 113673300 | 114187400 |
| 100392100 | 101961000 | 106323700 | 109819300 | 111773200 | 113138200 | 113684400 | 114203600 |
| 100479400 | 102346200 | 106590100 | 109944500 | 111852400 | 113279700 | 113694800 | 114215200 |
| 100529300 | 102578800 | 106662300 | 109949500 | 111997000 | 113416500 | 113697000 | 114441300 |
| 100570600 | 102811900 | 107601600 | 110174500 | 112101500 | 113420400 | 113698300 | 114458800 |
| 100575700 | 102906700 | 108033300 | 110178400 | 112226000 | 113532000 | 113707900 | 114471100 |
| 100596500 | 103155900 | 108233300 | 110193200 | 112288900 | 113544500 | 113709900 | |
| 101100600 | 103702300 | 108310100 | 110254600 | 112293800 | 113551300 | 113715800 | |
| 101185900 | 103951500 | 108413600 | 110481200 | 112623000 | 113551800 | 113731600 | |
| 101313000 | 104351000 | 108869700 | 110653400 | 112664300 | 113556700 | 113739100 | |

Pregnancy-specific chr18 u-DLRs

| | | | | | | | |
|---|---|---|---|---|---|---|---|
| 10164100 | 11127100 | 1225900 | 13326200 | 14966900 | 21431200 | 23230100 | 28368600 |
| 10230000 | 11280000 | 12431600 | 13421100 | 14970000 | 21579300 | 23449800 | 29048800 |
| 10248300 | 11283300 | 12561500 | 13431900 | 18700600 | 21587400 | 24037600 | 29144400 |
| 10263100 | 11378600 | 12565400 | 13432000 | 19028000 | 21668700 | 24125900 | 29926800 |
| 10272700 | 11378800 | 12723100 | 13497800 | 1911400 | 21709600 | 24318400 | 30488500 |
| 10433300 | 11532400 | 12741900 | 13511300 | 19222900 | 21972200 | 24360100 | 30722200 |
| 10563500 | 11750800 | 13135100 | 13517000 | 19273800 | 22278700 | 24421300 | 31581500 |
| 10706400 | 11759500 | 13226200 | 13527500 | 19294100 | 22307200 | 24459600 | 31941500 |
| 10723700 | 11802400 | 13246300 | 13625400 | 19898200 | 22733400 | 24709600 | 32154300 |
| 10842000 | 11817700 | 13247600 | 13627100 | 19991500 | 22783900 | 24873100 | |
| 1091900 | 11847900 | 13254200 | 13645500 | 20008200 | 22800300 | 25465700 | |
| 10936900 | 12035600 | 13270000 | 13647500 | 20815500 | 23006400 | 25734400 | |
| 11101200 | 12234800 | 13278200 | 14162800 | 20895400 | 23092300 | 28207100 | |

Tissue-specific chr13 u-DLRs

| | | | | | | | |
|---|---|---|---|---|---|---|---|
| 111000000 | 100286200 | 100395800 | 100591400 | 101203000 | 101763500 | 102608900 | 103229800 |
| 100057600 | 100291500 | 100406500 | 100596700 | 101212600 | 101820800 | 102732300 | 103236500 |
| 100066900 | 100315300 | 100442900 | 100656600 | 101288800 | 101825400 | 102775200 | 103354900 |
| 100078400 | 100318100 | 100446000 | 100689500 | 101314300 | 101885900 | 102852000 | 103365800 |
| 100080400 | 100328100 | 100456000 | 100704300 | 101334100 | 101931900 | 102901900 | 103400700 |
| 100097800 | 100341200 | 100463200 | 100932000 | 101391500 | 101996400 | 102906700 | 103427900 |
| 100110300 | 100344400 | 100479400 | 100989300 | 101404700 | 102271000 | 102979800 | 103430200 |
| 100122700 | 100358700 | 100541600 | 101034400 | 101425600 | 102293100 | 103044900 | 103539000 |
| 100140900 | 100375400 | 100557000 | 101045700 | 101593600 | 102397600 | 103045200 | 103547600 |
| 100142900 | 100375800 | 100559900 | 101097200 | 101593900 | 102498800 | 103094700 | |
| 100152100 | 100377300 | 100563200 | 101160600 | 101596900 | 102548800 | 103174600 | |
| 100172600 | 100387200 | 100565000 | 101194500 | 101605400 | 102558900 | 103202900 | |
| 100271900 | 100395500 | 100570600 | 101199900 | 101742200 | 102573800 | 103207500 | |

Tissue-specific chr18 u-DLRs

| | | | | | | | |
|---|---|---|---|---|---|---|---|
| 10004000 | 10218000 | 10366100 | 10472500 | 10723700 | 10825100 | 10890600 | 11037100 |
| 10007600 | 10218900 | 10369400 | 10472800 | 10724200 | 10828600 | 10902200 | 11049800 |
| 10010500 | 10220300 | 10372500 | 1047300 | 10730800 | 10834900 | 10925700 | 11054500 |
| 10013200 | 10230100 | 10373000 | 10500900 | 10731400 | 10838000 | 10936900 | 11058000 |
| 10029700 | 10231800 | 10381300 | 10571200 | 10734700 | 10838800 | 10971200 | 11063500 |
| 10030300 | 10263000 | 10398500 | 10582400 | 10737400 | 10844200 | 10986100 | 11068000 |
| 10035100 | 10272700 | 10403400 | 10592900 | 10745300 | 10844600 | 10990400 | 11071000 |
| 10052400 | 10287000 | 10404600 | 10682200 | 10755600 | 10845500 | 10993600 | 11074000 |
| 10068500 | 10300900 | 10408400 | 10682500 | 10774400 | 10847900 | 10994000 | 11099500 |
| 10100200 | 10301900 | 10410400 | 10703600 | 10775500 | 10864900 | 11009700 | |
| 10120900 | 10332200 | 10423100 | 10708300 | 10776700 | 10873300 | 11012000 | |
| 10153400 | 10343100 | 10433300 | 10717300 | 10785400 | 10878300 | 11021400 | |
| 10164600 | 10346600 | 10450400 | 10721900 | 10806500 | 10888800 | 11027200 | |

Pregnancy-specific chr13 hm-DLRs

| | | | | | | | |
|---|---|---|---|---|---|---|---|
| 100305000 | 107282100 | 114215200 | 28102000 | 43394700 | 46000500 | 50682600 | 92422900 |
| 100776700 | 110322500 | 20563600 | 28571600 | 44470900 | 47112300 | 50238200 | 99310800 |
| 107214300 | 111140600 | 21527500 | 28936500 | 45023500 | | | |

Pregnancy-specific chr18 hm-DLRs

| | | | | | | | |
|---|---|---|---|---|---|---|---|
| 24079700 | 3473100 | 56528700 | 72186200 | 9478700 | 3172300 | 35234800 | 61869500 |
| 73934900 | | | | | | | |

Tissue-specific chr13 hm-DLRs

| | | | | | | | |
|---|---|---|---|---|---|---|---|
| 111000000 | 100756100 | 101237300 | 101687300 | 101833600 | 102068100 | 102116200 | 103304300 |
| 100015300 | 100765100 | 101242700 | 101701700 | 101837400 | 102076800 | 102168800 | 103304500 |
| 100033700 | 100828300 | 101255600 | 101702000 | 101898100 | 102078200 | 102183900 | 103344400 |
| 100078000 | 100931800 | 101263700 | 101710300 | 101956100 | 102082400 | 102204700 | 103349700 |
| 100084900 | 100980400 | 101281000 | 101734400 | 101960500 | 102102200 | 102206000 | 103358800 |
| 100085400 | 100982800 | 101286200 | 101751100 | 101961200 | 102105900 | 102228800 | 103362900 |
| 100126100 | 101075300 | 101305600 | 101751500 | 101990200 | 102106400 | 102238000 | 103408800 |
| 100136900 | 101094300 | 101320900 | 101764100 | 101991600 | 102106800 | 102344000 | 103481700 |
| 100138400 | 101098400 | 101365300 | 101777000 | 101992300 | 102108000 | 102407700 | 105737500 |
| 100211300 | 101122000 | 101399900 | 101794700 | 102007400 | 102108700 | 102553800 | |
| 100231000 | 101182400 | 101451600 | 101796000 | 102052700 | 102109100 | 102580000 | |
| 100243900 | 101199800 | 101525100 | 101799300 | 102060500 | 102109800 | 103259500 | |
| 100589400 | 101202400 | 101533600 | 101831100 | 102060600 | 102112500 | 103265400 | |

Tissue-specific chr18 hm-DLRs

| | | | | | | | |
|---|---|---|---|---|---|---|---|
| 10018900 | 10207900 | 10935800 | 11956100 | 12326400 | 12659400 | 12855600 | 13622800 |
| 10020300 | 10373100 | 11208700 | 11971200 | 12367700 | 12660600 | 12871700 | 1379600 |
| 10030100 | 10377900 | 11274300 | 12027100 | 12375000 | 12734300 | 12908700 | 1399600 |
| 10034200 | 10547100 | 11571800 | 12027200 | 12389700 | 12738100 | 12908900 | 14090000 |
| 10045400 | 10560700 | 11690300 | 12231400 | 12443300 | 12738600 | 1296400 | 14975600 |
| 10046100 | 10710600 | 11807300 | 12251400 | 12463100 | 12738700 | 12969400 | 15021300 |
| 10052400 | 107600 | 11829700 | 12254300 | 12467300 | 12748100 | 12972900 | 18573300 |
| 10055600 | 10796000 | 11857200 | 12254900 | 12476900 | 12775300 | 12995300 | 18637700 |
| 10073000 | 10798100 | 11900 | 12255500 | 12521400 | 12782800 | 12996400 | 18710400 |
| 10093500 | 10799200 | 11912400 | 12282900 | 12547600 | 12788800 | 13137700 | |
| 10121200 | 10923400 | 11921600 | 12289600 | 12565700 | 12839200 | 1331300 | |
| 10169200 | 10927300 | 11947700 | 12301800 | 12641500 | 12849500 | 13608200 | |
| 10190600 | 10929600 | 11952800 | 12324700 | 12646700 | 12850200 | 13611300 | |

### NON PATENT LITERATURE

NPL1: Akolekar R, Beta J, Picciarelli G, Ogilvie C, D'Antonio F. Procedure-related risk of miscarriage following amniocentesis and chorionic villus sampling: a systematic review and meta-analysis. Ultrasound Obstet Gynecol. 2015 January; 45(1):16-26. doi: 10.1002/uog.14636.
NPL2: Chan K C, Zhang J, Hui A B, Wong N, Lau T K, Leung T N, Lo K W, Huang D W, Lo Y M. Size distributions of maternal and fetal DNA in maternal plasma. Clin Chem. 2004 January; 50(1):88-92.
NPL3: Chim S S, Jin S, Lee T Y, Lun F M, Lee W S, Chan L Y, Jin Y, Yang N, Tong Y K, Leung T Y, Lau T K, Ding C, Chiu R W, Lo Y M. Systematic search for placental DNA-methylation markers on chromosome 21: toward a maternal plasma-based epigenetic test for fetal trisomy 21. Clin Chem. 2008 March; 54(3):500-11.
NPL3: Chim S S, Tong Y K, Chiu R W, Lau T K, Leung T N, Chan L Y, Oudejans C B, Ding C, Lo Y M. Detection of the placental epigenetic signature of the maspin gene in maternal plasma. Proc Natl Acad Sci USA. 2005 Oct. 11; 102(41):14753-8.
NPL4: Chiu R W, Chan K C, Gao Y, Lau V Y, Zheng W, Leung T Y, Foo C H, Xie B, Tsui N B, Lun F M, Zee B C, Lau T K, Cantor C R, Lo Y M. Noninvasive prenatal diagnosis of fetal chromosomal aneuploidy by massively parallel genomic sequencing of DNA in maternal plasma. Version 2. Proc Natl Acad Sci USA. 2008 Dec. 23; 105(51):20458-63.
NPL5: Daniels G, Finning K, Martin P, Summers J. Fetal blood group genotyping: present and future. Ann N Y Acad Sci. 2006 September; 1075:88-95.
NPL6: Fan H C, Blumenfeld Y J, Chitkara U, Hudgins L, Quake S R. Analysis of the size distributions of fetal and maternal cell-free DNA by paired-end sequencing. Clin Chem. 2010 August; 56(8):1279-86.
NPL7: Gibas P, Narmontė M, Staševskij Z, Gordevičius J, Klimašauskas S, Kriukienė E. Precise genomic mapping of 5-hydroxymethylcytosine via covalent tether-directed sequencing. PLoS Biol. 2020 accepted
NPL8: Jensen T J, Kim S K, Zhu Z, Chin C, Gebhard C, Lu T, Deciu C, van den Boom D, Ehrich M. Whole genome bisulfite sequencing of cell-free DNA and its cellular contributors uncovers placenta hypomethylated domains. Genome Biol. 2015 Apr. 15; 16(1):78.
NPL9: Keravnou A, Ioannides M, Tsangaras K, Loizides C, Hadjidaniel M D, Papageorgiou E A, Kyriakou S, Antoniou P, Mina P, Achilleos A, Neofytou M, Kypri E, Sismani C, Koumbaris G, Patsalis P C. Whole-genome fetal and maternal DNA methylation analysis using MeDIP-NGS for the identification of differentially methylated regions. Genet Res (Camb). 2016 Nov. 11; 98:e15.
NPL10: Kriukienė E, Labrie V, Khare T, Urbanavičiūtė G, Lapinaitė A, Koncevičius K, Li D, Wang T, Pai S, Ptak C, Gordevičius J, Wang S C, Petronis A, Klimašauskas S. DNA unmethylome profiling by covalent capture of CpG sites. Nat Commun. 2013; 4:2190.
NPL11: Li Y, Zimmermann B, Rusterholz C, Kang A, Holzgreve W, Hahn S. Size separation of circulatory DNA in maternal plasma permits ready detection of fetal DNA polymorphisms. Clin Chem 2004; 50: 1002-11.
NPL12: Lo Y M, Chan K C, Sun H, Chen E Z, Jiang P, Lun F M, Zheng Y W, Leung T Y, Lau T K, Cantor C R, Chiu R W. Maternal plasma DNA sequencing reveals the genome-wide genetic and mutational profile of the fetus. Sci Transl Med. 2010 Dec. 8; 2(61):61ra91.
NPL13: Lo Y M, Chiu R W. Prenatal diagnosis: progress through plasma nucleic acids. Nat Rev Genet. 2007 January; 8(1):71-7.
NPL14: Lo Y M, Corbetta N, Chamberlain P F, Rai V, Sargent I L, Redman C W, Wainscoat J S. Presence of fetal DNA in maternal plasma and serum. Lancet 1997; 350:485-487.
NPL15: Lo Y M, Hjelm N M, Fidler C, Sargent I L, Murphy M F, Chamberlain P F, Poon P M, Redman C W, Wainscoat J S. Prenatal diagnosis of fetal RhD status by molecular analysis of maternal plasma. N Engl J Med. 1998 Dec. 10; 339(24):1734-8.
NPL16: Lun F M, Chiu R W, Sun K, Leung T Y, Jiang P, Chan K C, Sun H, Lo Y M. Noninvasive prenatal methylomic analysis by genomewide bisulfite sequencing of maternal plasma DNA. Clin Chem. 2013 November; 59(11):1583-94.
NPL17: Masevičius V, Nainytė M, Klimašauskas S. Synthesis of S-Adenosyl-L-Methionine Analogs with Extended Transferable Groups for Methyltransferase-Directed Labeling of DNA and RNA. Curr Protoc Nucleic Acid Chem. 2016 Mar. 1; 64:1.36.1-1.36.13.
NPL18: Old R W, Crea F, Puszyk W, Hulten M A. Candidate epigenetic biomarkers for non-invasive prenatal diagnosis of Down syndrome. Reprod Biomed Online. 2007 August; 15(2):227-35.
NPL19: Papageorgiou E A, Fiegler H, Rakyan V, Beck S, Hulten M, Lamnissou K, Carter N P, Patsalis P C. Sites of differential DNA methylation between placenta and peripheral blood: molecular markers for noninvasive prenatal diagnosis of aneuploidies. Am J Pathol. 2009 May; 174(5):1609-18.
NPL20: Parker S E, Mai C T, Canfield M A, Rickard R, Wang Y, Meyer R E, Anderson P, Mason C A, Collins J S, Kirby R S, Correa A; National Birth Defects Prevention Network. Updated National Birth Prevalence estimates for selected birth defects in the United States, 2004-2006. Birth Defects Res A Clin Mol Teratol. 2010 December; 88(12):1008-16.
NPL21: Song C X, Szulwach K E, Fu Y, Dai Q, Yi C, Li X, Li Y, Chen C H, Zhang W, Jian X, Wang J, Zhang L, Looney T J, Zhang B, Godley L A, Hicks L M, Lahn B T, Jin P, He C. Selective chemical labeling reveals the genome-wide distribution of 5-hydroxymethylcytosine. Nat Biotechnol. 2011 January; 29(1):68-72.
NPL22: Staševskij Z, Gibas P, Gordevičius J, Kriukienė E, Klimašauskas S. Tethered Oligonucleotide-Primed Sequencing, TOP-Seq: A High-Resolution Economical Approach for DNA Epigenome Profiling. Mol Cell. 2017 Feb. 2; 65(3):554-564.e6.
NPL23: Tong Y K, Jin S, Chiu R W, Ding C, Chan K C, Leung T Y, Yu L, Lau T K, Lo Y M. Noninvasive prenatal detection of trisomy 21 by an epigenetic-genetic chromosome-dosage approach. Clin Chem. 2010 January; 56(1):90-8.
NPL24: Tsaliki E, Papageorgiou E A, Spyrou C, Koumbaris G, Kypri E, Kyriakou S, Sotiriou C, Touvana E, Keravnou A, Karagrigoriou A, Lamnissou K, Velissariou V, Patsalis P C. MeDIP real-time qPCR of maternal peripheral blood reliably identifies trisomy 21. Prenat Diagn. 2012 October; 32(10):996-1001.
NPL25: Weber M, Davies J J, Wittig D, Oakeley E J, Haase M, Lam W L, Schübeler D. Chromosome-wide and promoter-specific analyses identify sites of differential DNA methylation in normal and transformed human cells. Nat Genet. 2005 August; 37(8):853-62.
NPL26: Zimmermann B, Holzgreve W, Wenzel F, Hahn S. Novel real-time quantitative PCR test for trisomy 21. Clin Chem. 2002 February; 48(2):362-3.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A method for prenatal diagnosis of a trisomy 21 and/or fetal gender using a sample of isolated cfDNA, the method comprising:
a) enzymatic covalent labeling of nucleic acid molecules of the sample of isolated cfDNA at unmodified CG, uCG, sites with eM.Sssl methyltransferase or hydroxymethylated CG, hmCG, sites with beta-glucosyltransferase and derivatization of said labels with DNA oligonucleotide (ODN);
b) measuring the level of the labeled CG-containing regions of the sample of isolated cfDNA at one or more regions of chromosomal DNA from the human genome shown in Tables 4, 5, and 6;
c) comparing the measured level of the labeled CG-containing regions of step b) to a standard reference value of at least one region from Table 4, 5, or 6.

13. The method of claim 12, comprising diagnosing a trisomy based on said comparison of step c), wherein trisomy 21 is diagnosed if the measured level of the labeled CG-containing regions of step b) is (i) higher than the standard reference value from a woman bearing a fetus without trisomy 21; or (ii) lower than the standard reference value from a woman bearing a fetus without trisomy 21; or (iii) comparable to the standard reference value from a woman bearing a fetus with trisomy 21.

14. The method of claim 12, comprising detecting fetal gender based on said comparison of step c), wherein female gender of a fetus is detected if the measured level of the labeled CG-containing regions of step b) is comparable to the standard reference value from a woman bearing a female fetus, and male gender of a fetus is detected if the measured level of the regions of step b) is comparable to the standard reference value from a woman bearing a male fetus.

15. The method of claim 12, wherein the levels of the labeled CG-containing regions in the sample of isolated cfDNA are measured by real time quantitative polymerase chain reaction (qPCR).

16. The method of claim 12, wherein the level of at least one labeled CG from any labeled CG-containing region shown in Tables 4, 5 and 6 is measured by qPCR.

17. The method of claim 12, further comprising producing nucleic acid molecules from the labeled CG-containing regions using a nucleic acid polymerase which contacts the labeled nucleic acid sequence at or around the site of the labeled uCG or hmCG; wherein polymerization starts from the 3′-end of an oligonucleotide primer non-covalently attached to the ODN of the labeled CG-containing region of the sample of isolated cfDNA; for further amplification of labeled CG-containing regions of the sample of isolated cfDNA, an oligonucleotide primer non-covalently attached to the ODN and yet another oligonucleotide primer that binds to the one strand of an adapter sequence attached to the labeled CG-containing regions through ligation-mediated PCR are used to obtain a sample enriched in unmodified or hydroxymethylated DNA.

18. The method of claim 12, further comprising producing nucleic acid molecules from the labeled CG-containing regions of the sample of isolated cfDNA using a nucleic acid polymerase which contacts the labeled CG-containing regions at or around the site of labeled uCG or hmCG; wherein polymerization starts from the 3′-end of a primer non-covalently attached to the ODN of the labeled CG-containing regions and partially to genomic nucleotides near the labeled CG sites and another primer binds to genomic region near the labeled CG sites.

19. The method of claim 17, wherein the levels of the labeled CG-containing regions in the sample of isolated cfDNA are measured by real time quantitative polymerase chain reaction (qPCR) or sequencing.

20. The method of claim 17, wherein one or more sets of oligonucleotide primers selected from SEQ ID 1-18 are used.

21. A kit comprising the oligonucleotide primers of claim 20 and an enzyme for uCG and hmC labeling for covalent labeling and enrichment of uCG and hmC sites.

22. The kit of claim 21, further comprising DNA oligonucleotide (ODN) for derivatization of the labeled uCG and hmC sites.

23. The kit of claim 21, which further comprises oligonucleotide adaptors and oligonucleotide primers for the ODN-directed and in part by ligation mediated amplification of the labeled regions.

24. The method of claim 18, wherein the levels of the labeled CG-containing regions in the sample of isolated cfDNA are measured by real time quantitative polymerase chain reaction (qPCR) or sequencing.

25. The method of claim 18, wherein one or more sets of oligonucleotide primers selected from SEQ ID 1-18 are used.

26. A kit comprising the oligonucleotide primers of claim 25 and an enzyme for uCG and hmC labeling for covalent labeling and enrichment of uCG and hmC sites.

27. The kit of claim 26, further comprising DNA oligonucleotide (ODN) for derivatization of the labeled uCG and hmC sites.


SEQUENCE LISTING (18 sequences)

SEQ ID NO: 1 - DNA, 20 bp, Artificial Sequence
  Primer 1
caactccctacagccccttg

SEQ ID NO: 2 - DNA, 22 bp, Artificial Sequence
  Primer 2
aaattgcatgattcccctgaca

SEQ ID NO: 3 - DNA, 26 bp, Artificial Sequence
  Primer 3
atgactggcttatttcacttagcatc

SEQ ID NO: 4 - DNA, 23 bp, Artificial Sequence
  Primer 4
agtcctgctatatgcaacacctt

SEQ ID NO: 5 - DNA, 28 bp, Artificial Sequence
  Primer 5
ggtatttacaaaagtctgcaccttagtc

SEQ ID NO: 6 - DNA, 20 bp, Artificial Sequence
  Primer 6
ctgccaacttcacccagagt

SEQ ID NO: 7 - DNA, 26 bp, Artificial Sequence
  Primer 7
tagaaatctttaggaggtggtgaatg

SEQ ID NO: 8 - DNA, 19 bp, Artificial Sequence
  Primer 8
catggtggaagagatgggc

SEQ ID NO: 9 - DNA, 20 bp, Artificial Sequence
  Primer 9
gcagaggttgcagtgagctg

SEQ ID NO: 10 - DNA, 22 bp, Artificial Sequence
  Primer 10
gtctggatgcaaaaatcccttt

SEQ ID NO: 11 - DNA, 21 bp, Artificial Sequence
  Primer 11
gctgtccctgtggttaaggtc

SEQ ID NO: 12 - DNA, 18 bp, Artificial Sequence
  Primer 12
gccaccacaacagcacca

SEQ ID NO: 13 - DNA, 20 bp, Artificial Sequence
  Primer 13
ccccatcaccaacttcactc

SEQ ID NO: 14 - DNA, 21 bp, Artificial Sequence
  Primer 14
gaaactgagtctctcgcaagg

SEQ ID NO: 15 - DNA, 45 bp, Artificial Sequence
  Primer 15
cctctctatgggcagtcggtgattgacctgcttcctgtgttgagc

SEQ ID NO: 16 - DNA, 45 bp, Artificial Sequence
  Primer 16
tgttacatctggtagtcagtctccaaaccacacaaaaaagtggag

SEQ ID NO: 17 - DNA, 30 bp, Artificial Sequence
  Primer 17
gtagaaaaggggaagaaaagtagaaacagc

SEQ ID NO: 18 - DNA, 45 bp, Artificial Sequence
  Primer 18
tgttacatctggtagtcagtctccaaaccacacaaaaaagcccct